(19) United States
(12) Patent Application Publication
(10) Pub. No.: US 2012/0203290 A1
(43) Pub. Date: Aug. 9, 2012

(54) METHOD AND APPARATUS FOR SPINAL FIXATION

(75) Inventors: Christopher Warren, Aliso Viejo, CA (US); Robert Flower, Sun City, CA (US)

(73) Assignee: INTERVENTIONAL SPINE, INC., Irvine, CA (US)

(21) Appl. No.: 13/365,792

(22) Filed: Feb. 3, 2012

Related U.S. Application Data
(60) Provisional application No. 61/439,798, filed on Feb. 4, 2011.

(51) Int. Cl. A61B 17/56

(52) U.S. Cl. 606/86 A

(57) ABSTRACT

Fusion of cervical spinal vertebrae with one or more fixation devices can be accomplished with the described tools and methods. For example, a guidewire introducer can include a tubular introducer cannula and a handle. The handle can be angularly offset from the introducer cannula such that positioning of the introducer on the cervical spine does not interfere with a patient's head. A sheath assembly can include inner and outer sheath bodies and a handle. The handle is angularly offset from the sheath bodies such that the sheath assembly can be applied to the cervical spine without interference to the patient's head. The sheath body can be curved or straight. Various tools such as drills, tapping devices, compression tools, and pin release tools can be applied to the cervical spine through the sheath body to apply the fixation device. The tools can include elongate flexible shafts.

### PRIORITY

The present application claims the benefit of U.S. Provisional Application No. 61/439,798, filed Feb. 4, 2011, the disclosure of which is hereby incorporated by reference herein in its entirety.

### BACKGROUND OF THE INVENTION

1. Field of the Invention

The present application relates to medical devices and, more particularly, to methods and apparatus for spinal stabilization.

2. Description of the Related Art

The human spine is a flexible weight bearing column formed from a plurality of bones called vertebrae. There are thirty three vertebrae, which can be grouped into five regions (cervical, thoracic, lumbar, sacral, and coccygeal). Moving down the spine, there are generally seven cervical vertebra, twelve thoracic vertebra, five lumbar vertebra, five sacral vertebra, and four coccygeal vertebra. The vertebra of the cervical, thoracic, and lumbar regions of the spine are typically separate throughout the life of an individual. In contrast, the vertebra of the sacral and coccygeal regions in an adult are fused to form two bones, the five sacral vertebra which form the sacrum and the four coccygeal vertebra which form the coccyx.

In general, each vertebra contains an anterior, solid segment or body and a posterior segment or arch. The arch is generally formed of two pedicles and two laminae, supporting seven processes—four articular, two transverse, and one spinous. There are exceptions to these general characteristics of a vertebra. For example, the first cervical vertebra (atlas vertebra) has neither a body nor spinous process. In addition, the second cervical vertebra (axis vertebra) has an odontoid process, which is a strong, prominent process, shaped like a tooth, rising perpendicularly from the upper surface of the body of the axis vertebra. Further details regarding the construction of the spine may be found in such common references as Gray's Anatomy, Crown Publishers, Inc., 1977, pp. 33-54, which is herein incorporated by reference.

The human vertebrae and associated connective elements are subjected to a variety of diseases and conditions which cause pain and disability. Among these diseases and conditions are spondylosis, spondylolisthesis, vertebral instability, spinal stenosis and degenerated, herniated, or degenerated and herniated intervertebral discs. Additionally, the vertebrae and associated connective elements are subject to injuries, including fractures and torn ligaments and surgical manipulations, including laminectomies.

The pain and disability related to the diseases and conditions often result from the displacement of all or part of a vertebra from the remainder of the vertebral column. Over the past two decades, a variety of methods have been developed to restore the displaced vertebra to their normal position and to fix them within the vertebral column. Spinal fusion is one such method. In spinal fusion, one or more of the vertebra of the spine are united together (“fused”) so that motion no longer occurs between them. The vertebra may be united with various types of fixation systems. These fixation systems may include a variety of longitudinal elements such as rods or plates that span two or more vertebrae and are affixed to the vertebrae by various fixation elements such as wires, staples, and screws (often inserted through the pedicles of the vertebrae). These systems may be affixed to either the posterior or the anterior side of the spine. In other applications, one or more bone screws may be inserted through adjacent vertebrae to provide stabilization.

U.S. Patent Publication 2004/0127906 (U.S. patent application Ser. No. 10/623,193, filed Jul. 18, 2003) entitled “METHOD AND APPARATUS FOR SPINAL FUSION” describes a bone fixation screw and technique used to secure two adjacent vertebra to each other in trans-laminar, trans-facet or facet-pedicle (e.g., the Boucher technique) applications. This publication is incorporated herein by reference in its entirety. For example, in a trans-facet application, the fixation device extends through a facet of a first vertebra and into the facet of a second, typically inferior, vertebra. In a trans-laminar application, screws, the fixation device, extend through the spinous process and facet of a first vertebra and into the facet of a second, typically inferior, vertebra. In a facet-pedicle application (e.g., the Boucher technique), the fixation device extends through the facet of a first vertebra and into the pedicle a second, typically inferior, vertebra. These procedures are typically (but not necessarily) preformed with bilateral symmetry.

Notwithstanding the success of the above described devices and methods, there are certain challenges associated with applying the trans-laminar, trans-facet or facet-pedicle (e.g., the Boucher technique) techniques to the cervical portion of the vertebrae. For example, due to the anatomy of the cervical region and interference due to the back of the head in a trans-facet approach, the fixation device may need to extend along an axis that, when extended, interferes with the back of the patient's head. For example, FIG. 1 illustrates a portion of the cervical region and a cannulated access device, which extends over the desired entry axis of the fixation device (not shown). As shown, the back of the patient's spine can interfere with the insertion of the fixation device and the various tools needed to insert the fixation device. While U.S. Pat. No. 7,938,832, which is hereby incorporated by reference in its entirety, provides many solutions to the challenges discussed above, additional improvement would further enhance such techniques.

### SUMMARY OF THE INVENTION

In some embodiments, a device used for deploying a spinal fixation device comprises an elongated cannulated member and a handle. The elongated cannulated member has a proximal end, a distal end, a first longitudinal axis extending therebetween, and an outer surface. The cannulated member comprises an elongated opening on the outer surface. The handle extends along a second longitudinal axis. The first and second longitudinal axis form an angle with respect to each other. The elongated opening is configured to receive an elongate tubular member having a third longitudinal axis when the third longitudinal axis is oriented transversely to the first longitudinal axis.

In various embodiments, a wire introducer for creating a tissue track for a guidewire, comprises an elongated cannulated member, a handle, and a trocar. The elongated cannulated member has a first longitudinal axis, a distal end and a proximal end, the distal end including at least one cutting element. The handle extends along a second longitudinal axis, wherein the first and second longitudinal axes form an angle with respect to each other. The trocar has a distal end with a sharpened tip and a proximal end configured to receive a strike pin. The trocar is positioned within the cannulated member such that the distal end and proximal end extend beyond the elongated cannulated member.

In some embodiments, a system for coupling a first superior vertebra of a cervical spine to a second inferior vertebra comprises a fixation device and an elongated tubular device. The fixation device has a distal end and a proximal end. The distal end of the fixation device is configured to extend between the first superior vertebra and the second inferior vertebra. The elongated tubular device is configured to apply the fixation device. The tubular device has a first longitudinal axis and a handle extending along a second longitudinal axis. The first and second longitudinal axes form an angle with respect to each other such that when the elongated tubular device is applied to the cervical spine from a direction above the cervical spine, the fixation device can be applied without interference from the head of the patient.

In some embodiments, a system for establishing access for a fixation device configured to extend between a first superior vertebra of a cervical spine to a second inferior vertebra comprises an elongated tubular device and an elongated flexible member. The elongated tubular device has a first longitudinal axis and a handle extending along a second longitudinal axis, the first and second longitudinal axis form an angle with respect to each other. The elongated flexible member has a distal end and a proximal end. The distal end of the device is coupled to a tool, and the proximal end of the device is coupled to a handle.

In some embodiments, a device used for deploying a spinal fixation device comprises an elongated flexible transmission member, a tool, and a handle. The elongated flexible transmission member has a distal end and a proximal end. The tool is coupled to the distal end of the transmission member. The handle is coupled to the proximal end of the transmission member.

In some embodiments, a method of providing spinal fixation in a cervical spine comprises advancing a distal end of an elongated cannulated member, removing the trocar, advancing a first guidewire, removing the first guidewire, advancing a second guidewire, removing the elongated cannulated member, advancing a fascia cutter over the second guidewire, cutting the patient's fascia, removing the fascia cutter, advancing a dilation device, and inserting a distal end of a fixation device. The distal end of the elongated cannula member is advanced with a trocar positioned therein to a first, superior vertebra in the cervical spine to establish a tissue tract. The trocar is removed from the elongated cannulated member. The first guidewire is advanced though the elongated cannulated member and at least partially into the first vertebra. The first guidewire is removed from the elongated cannulated member. The second guidewire is advanced through the elongated cannulated member. The patient's fascia is cut with the fascia cutter. The dilation device is advanced over the second guidewire. The distal end of the fixation device is inserted through the dilation device and through the first vertebra and into the second vertebra.

In some embodiments, a device used for deploying a spinal fixation device comprises an elongated cannulated member and a handle. The elongated cannulated member has a first longitudinal axis. The handle extends away from the elongated cannulated member along second longitudinal axis. The handle includes a gripping portion.

In some embodiments, a method of placing a guidewire near a cervical portion of the spine comprises advancing an elongated member along a first longitudinal axis extending from the cervical portion of the spine toward the head of the patient while grasping a handle coupled to the elongated member and located angularly offset from the elongated member; and inserting a guidewire through the elongated member.

In some embodiments, a method of inserting a fixation device through a first superior vertebra and into a second inferior vertebra in a cervical portion of the spine comprises advancing a fixation device, advancing the bone anchor of the fixation device, preoximally retracting the body of the fixation device, advancing a second fixation device, advancing the bone anchor of the second fixation device, advancing a second proximal anchor, and retracting the body of the second fixation device. A fixation device that comprises a body having a first portion that forms a first bone anchor and a second portion that forms a proximal end through a cannulated member and through a portion of the first cervical vertebra is advanced. The bone anchor of the fixation device is advanced into the second cervical vertebra. The proximal anchor is advanced distally along the fixation device. The body of the fixation device is retracted proximally with respect to the proximal anchor to adjust compression across the first and second cervical vertebra. with substantially bilateral symmetry, a second fixation device is advanced that comprises a body having a first portion that forms a second bone anchor and a second portion that forms a proximal end through a second cannulated member and through a portion of the first vertebra. The bone anchor of the second fixation device is advanced into the second vertebra. The second proximal anchor is advanced distally along the second fixation device. The body of the second fixation device is retracted proximally with respect to the proximal anchor to adjust compression across the first and second vertebrae.

In some embodiments, a fascia cutter for cutting fascia surrounding a portion of the spine comprises an elongated body and a plurality of cutting elements. The elongated body has a proximal end, a distal end and a lumen extending therethrough. The lumen has a distal opening at the distal end and a proximal opening at the proximal end. The plurality of cutting elements is positioned on the distal end of the elongated body. Each of the plurality of cutting elements defines a cutting edge that extends generally radially from the distal end of the lumen.

In some embodiments, a method of providing access to a portion of a spine, comprises advancing a guidewire and advancing a fascia cutter. The guidewire is advanced posteriorly through a patient's tissue to a first vertebra. The fascia cutter comprises at least one sharpened element and is advanced over the guidewire and towards the first vertebra to cut the patient's fascia.

In some embodiments, a method of coupling a first superior vertebra to a second inferior vertebra, comprises advancing a first guidewire, removing the first guidewire, and advancing a second guidewire. The first guidewire is advanced with a generally sharpened distal tip into the first vertebra and into the second vertebra along a first insertion axis. The second guidewire with a generally blunt distal tip is advanced along the first insertion axis into the second vertebra and through a hole created by the first guidewire.

### BRIEF DESCRIPTION OF THE DRAWINGS

FIG. 1 is a side elevational view of a cervical spine having a fixation device extending across facets of two adjacent vertebrae.

FIG. 2 is a posterior view of the cervical spine of FIG. 1.

FIG. 3A is a side perspective view of an embodiment of the fixation device of FIGS. 1 and 2.

FIG. 3B is a side view of the fixation device of FIG. 3A

FIG. 3C is a cross-sectional view taken through line 3C-3C of FIG. 3B.

FIG. 4 is a side elevational view of the cervical spine and an embodiment of a wire introducer.

FIG. 5 is a side elevational view of the cervical spine and the wire introducer of FIG. 4 with an embodiment of a strike pin coupled thereto.

FIG. 6 is a side elevational view of the cervical spine and an alternative embodiment of a wire introducer with a trocar inserted therein and an alternative embodiment of a strike pin coupled thereto.

FIG. 7 is a side elevational view of the cervical spine and the wire introducer of FIG. 4 with an embodiment of sharp guidewire inserted therein.

FIG. 8 is a side elevational view of the wire introducer of FIG. 4 with an embodiment of a drill stop attached thereto.

FIG. 9 is a side elevational view of the cervical spine and the wire introducer of FIG. 4 with an embodiment of blunt guidewire inserted therein.

FIG. 10 is a side elevational view of the cervical spine with the blunt guidewire of FIG. 9 and the wire introducer of FIG. 4 removed.

FIG. 11 is a side elevational view of the cervical spine with the guidewire of FIG. 9 and an embodiment of a fascia cutter inserted over the guidewire.

FIG. 12 is a side elevational view of the cervical spine with the guidewire of FIG. 9 and an embodiment of a sheath assembly in a first position.

FIG. 13 is a side elevational view of the cervical spine with the guidewire of FIG. 9 and an embodiment of the sheath assembly of FIG. 12 in a second position with a center portion removed.

FIG. 14 is a side elevational view of the cervical spine with the guidewire of FIG. 9 and the sheath assembly of FIG. 12 with a drill inserted therein.

FIG. 15 is a side elevational view of the cervical spine with the guidewire of FIG. 9 and the sheath assembly of FIG. 12 with a tapping device inserted therein.

FIG. 16 is a side elevational view of the cervical spine with the guidewire of FIG. 9 and the sheath assembly of FIG. 12 with a driving device inserted therein.

FIG. 17 is a side elevational view of the cervical spine with the guidewire of FIG. 9 and the sheath assembly of FIG. 12 with a compression device inserted therein.

FIG. 18 is a side elevational view of the cervical spine with the guidewire of FIG. 9 and the sheath assembly of FIG. 12 with a pin removal device inserted therein.

FIG. 19A is a side view of the wire introducer of FIG. 4.

FIG. 19B is a top view of a cannula portion of the wire introducer of FIG. 4.

FIG. 19C is a cross-sectional side view of the cannula portion of FIG. 19B.

FIG. 19D is a close-up side view of the distal end of the cannula portion of FIG. 19B.

FIG. 20A is a cross-sectional side view of the strike pin of FIG. 5.

FIG. 20B is a close-up side view of the distal end of the strike pin of FIG. 5.

FIG. 21A is a side view of the alternative embodiment of the wire introducer of FIG. 6.

FIG. 21B is a side view of a cannula portion of the wire introducer of FIG. 21A.

FIG. 21C is a cross-sectional view of a cannula portion of the wire introducer of FIG. 21A.

FIG. 21D is a front view of a cannula portion of the wire introducer of FIG. 21A.

FIG. 21E is a side view of a proximal end of a cannula portion of the wire introducer of FIG. 21A.

FIG. 21F is a side view of a distal end of a cannula portion of the wire introducer of FIG. 21A.

FIG. 21G is a rear view of a cannula portion of the wire introducer of FIG. 21A.

FIG. 22A is a side view of a trocar of the wire introducer of FIG. 21A.

FIG. 22B is a side perspective view of a trocar connecting hub of the wire introducer of FIG. 21A.

FIG. 22C is a cross-sectional view of a trocar connecting hub of the wire introducer of FIG. 21A.

FIG. 22D is a side view of a trocar of the wire introducer of FIG. 21A.

FIG. 23A is a perspective view of an alternative embodiment of a strike pin of FIG. 6.

FIG. 23B is a side view of the strike pin of FIG. 23A.

FIG. 23C is an enlarged view of a portion of the strike pin of FIG. 23A.

FIG. 24A is a side view of the sharp guidewire of FIG. 7.

FIG. 24B is an enlarged view of a portion of the sharp guidewire of FIG. 24A.

FIG. 25A is a top view of a drill stop of FIG. 8.

FIG. 25B is a cross-sectional side view of the drill stop of FIG. 25A.

FIG. 26 is a side view of the blunt guidewire of FIG. 9.

FIG. 27A is a perspective view of the fascia cutter of FIG. 11.

FIG. 27B is a side view of the fascia cutter of FIG. 27A.

FIG. 27C is a front view of the fascia cutter of FIG. 27A.

FIG. 27D is a cross-sectional view of the fascia cutter of FIG. 27A.

FIG. 27E is an enlarged view of a distal end of the fascia cutter of FIG. 27A.

FIG. 28A is a side view of the sheath assembly of FIG. 12.

FIG. 28B is a side view of an inner sheath of the sheath assembly of FIG. 28A.

FIG. 28C is a side view of an outer sheath of the sheath assembly of FIG. 28A.

FIG. 29 is a cross-sectional side view of the drill of FIG. 14.

FIG. 30A is a perspective view of a drilling element of the drill of FIG. 29.

FIG. 30B is a side view of a drilling element of the drill of FIG. 29.

FIG. 30C is a front view of a drilling element of the drill of FIG. 29.

FIG. 30D is a cross-sectional view of a drilling element of the drill of FIG. 29.

FIG. 31 is a side view of a handle device.

FIG. 32A is a side view of a transmission member of the drill of FIG. 29.

FIG. 32B is a cross-sectional view of a transmission member of the drill of FIG. 29.

FIG. 32C is an enlarged view of an embodiment of cut pattern of the transmission member of the drill of FIG. 29.

FIG. 33A is a side view of the tapping device of FIG. 15.

FIG. 33B is a cross-sectional view of the tapping element of the tapping device of FIG. 33A.

FIG. 33C is a front view of the tapping device of FIG. 33A.

FIG. 34A is a cross-sectional view of the driving device of FIG. 16

FIG. 34B is a close-up perspective view of the driving element of the driving device of FIG. 34A.

FIG. 35 is a cross-sectional view of the compression device of FIG. 17.

FIG. 36A is a perspective view of a distal cap of the compression device of FIG. 35.

FIG. 36B is a cross-sectional view of the distal cap of FIG. 36A.

FIG. 37A is a cross-sectional side view of a tensioner member of the compression device of FIG. 35.

FIG. 37B is an enlarged view of an embodiment of a cut pattern of the tensioner member of FIG. 37A.

FIG. 38A is a perspective view of a collet of the compression device of FIG. 35.

FIG. 38B is a front view of the collet of FIG. 38A.

FIG. 38C is a cross-sectional view of the collet of FIG. 38A.

FIG. 39A is a cross-sectional view of a distal housing of the compression device of FIG. 35.

FIG. 39B is an enlarged view of an embodiment of a cut pattern of the distal housing member of FIG. 39A.

FIG. 40 is a cross-sectional view of the collet and distal cap of the compression device of FIG. 35.

FIG. 41A is a perspective view of a connector shaft of the compression device of FIG. 35.

FIG. 41B is a cross-sectional view of the connector shaft of FIG. 41A.

FIG. 42A is a perspective view of a proximal portion of the compression device of FIG. 35.

FIG. 42B is a cross-sectional view of a proximal portion of the compression device of FIG. 35.

FIG. 43A is a cross-sectional view of a pin remover device of FIG. 18.

FIG. 43B is an enlarged cross-sectional view of a portion of the pin remover device of FIG. 43A.

FIG. 44A is a perspective view of an alternative embodiment of a tensioner member of the compression device of FIG. 35.

FIG. 44B is a cross-sectional side view of the tensioner member of FIG. 44A.

FIG. 45 is a cross-sectional side view of an embodiment of a funnel and push rod.

### DETAILED DESCRIPTION OF THE PREFERRED EMBODIMENTS

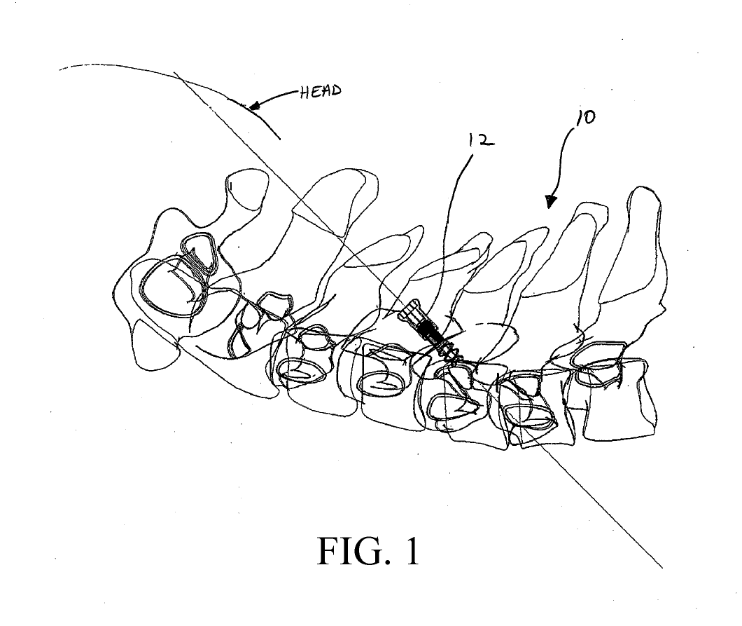

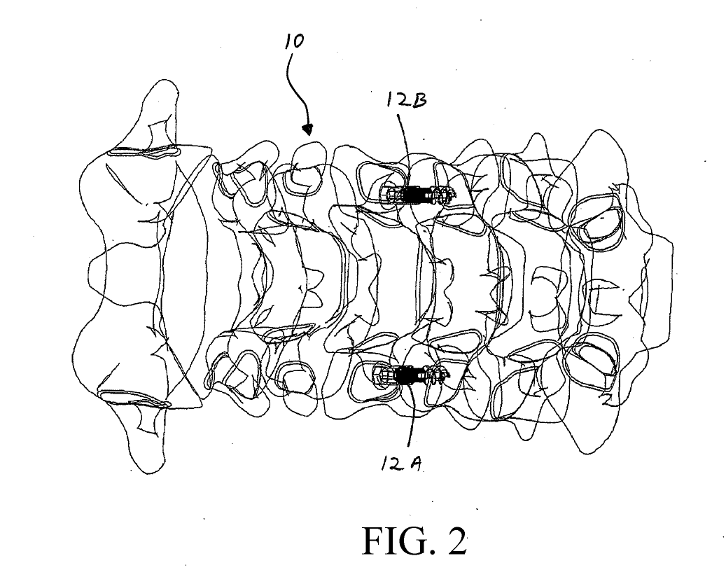

Referring to FIG. 1, a side elevational view of an embodiment of the cervical portion of the spine 10 with a fixation device 12 that extends across the facet joint of two adjacent vertebrae (i.e., a trans-facet application) is illustrated. With reference to FIG. 2, a pair of bone fixation devices 12A, 12B can preferably (but not necessarily) be used with substantial bilateral symmetry to secure two adjacent vertebra to each other. In FIGS. 1 and 2 the bone fixation device is highlighted such that the portions hidden by the vertebrae can be seen. In this manner, the adjacent vertebrae of the spine are united together (“fused”) so that motion no longer occurs between the vertebrae. Thus, even in the absence of a stabilizing bar tying pedicle screws to adjacent vertebrae, the fixation devices 12A, 12B can be used to stabilize two vertebrae to each other pending the healing of a fusion. See also U.S. Patent Publication No. 2004/0127905, filed Jul. 18, 2003, application Ser. No. 10/623,193, which is incorporated by reference herein in its entirety.

The disclosure herein will focus on a method of fusing two adjacent vertebrae together, as described above. However, it should be appreciated that certain aspects of the devices and methods described herein can find applications in other systems for stabilizing and/or fixating the spine. For example, such fixation systems may include a variety of longitudinal elements such as rods or plates that span two or more vertebrae and are affixed to the vertebrae by various fixation elements such as wires, staples, and screws (often inserted through the pedicles of the vertebrae). These systems may be affixed to either the posterior or the anterior side of the spine. Certain aspects and features of the devices and methods disclosed herein can also find utility when stabilizing/fixing other areas of the spine (e.g., lumbar spine).

Anchor Device

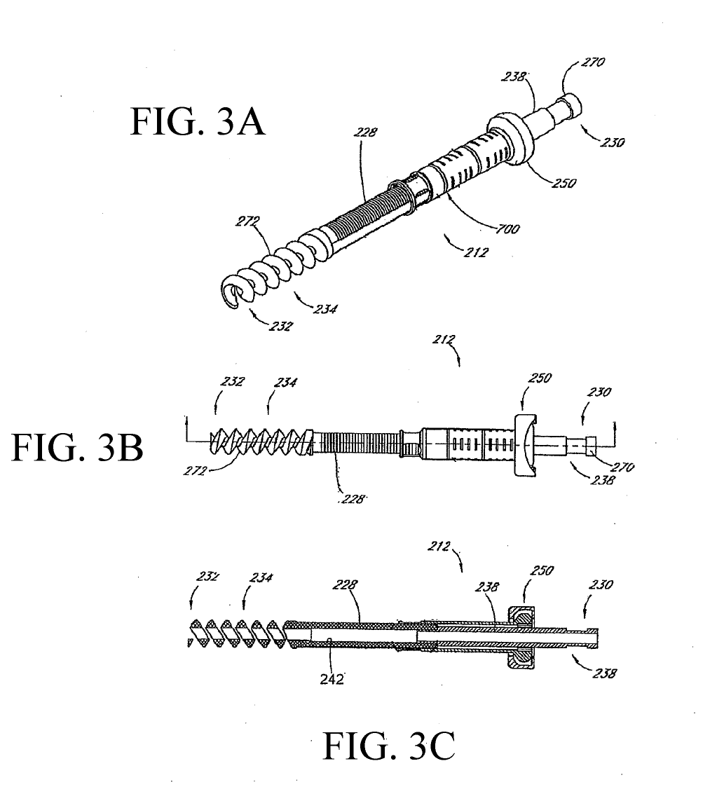

FIGS. 3A-C illustrate an embodiment of a bone fixation device 212 that can be used in the method described herein. As will be apparent from the description below, the illustrated bone fixation device 212 is particularly advantageous for spinal fixation. The device 212 comprises the body 228 that extends between a proximal end 230 and the distal end 232. The length, diameter and construction materials of the body 228 can be varied, depending upon the intended clinical application. In embodiments optimized for spinal stabilization in the cervical spine 10 (FIGS. 1-2) in an adult human population, the body 228 will generally be within the range of from about 10-20 mm in length and within the range of from about 2.5-4 mm in maximum diameter. The length of the helical distal anchor 234, discussed below, may be about 3-15 millimeters. Of course, it is understood that these dimensions are illustrative and that they may be varied as required for a particular patient or procedure.

The distal end 232 of the body 228 is provided with the cancellous bone anchor and/or distal cortical bone anchor 234. Generally, for spinal stabilization, the distal bone anchor 234 is adapted to be rotationally inserted into and through a portion (e.g., the facet) of a first, superior, vertebra and then into a portion (e.g., a facet) of a second, inferior vertebra. In the illustrated embodiment, the distal anchor 234 comprises a helical locking structure 272 for engaging cancellous and/or distal cortical bone. In the illustrated embodiment, the locking structure 272 comprises a flange that is wrapped around a central core, which in the illustrated embodiment is generally cylindrical in shape. The flange 272 extends through at least one and generally from about two to about 50 or more full revolutions depending upon the axial length of the distal anchor 234 and intended application. The flange will generally complete from about 2 to about 60 revolutions. The helical flange 272 is preferably provided with a pitch and an axial spacing to optimize the retention force within cancellous bone. While the helical locking structure 272 is generally preferred for the distal anchor, it should be appreciated that in modified embodiments other types of anchors could be used to secure the device in the cancellous bone anchor and/or distal cortical bone, such as, for example, various combinations and sub-combinations of hooks, prongs, expandable flanges, etc.

The helical flange 272 of the illustrated embodiment has a generally triangular cross-sectional shape. However, it should be appreciated that the helical flange 272 can have any of a variety of cross sectional shapes, such as rectangular, oval or other as deemed desirable for a particular application through routine experimentation in view of the disclosure herein. For example, in one modified embodiment, the flange 272 has a triangular cross-sectional shape with a blunted or square apex. Particularly advantageous cross-sectional shapes of the flange are the blunted or square type shapes. Such shapes can reduce cutting into the bone as the proximal end of the device is activated against causing a windshield wiper effect that can loosen the device 212. The outer edge of the helical flange 272 defines an outer boundary. The ratio of the diameter of the outer boundary to the diameter of the central core can be optimized with respect to the desired retention force within the cancellous bone and giving due consideration to the structural integrity and strength of the distal anchor 234. Another aspect of the distal anchor 234 that can be optimized is the shape of the outer boundary and the central core, which in the illustrated embodiment are generally cylindrical.

The distal end 232 and/or the outer edges of the helical flange 272 can be atraumatic (e.g., blunt or soft). This inhibits the tendency of the stabilization device 212 to migrate anatomically distally and potentially out of the vertebrae after implantation. Distal migration is also inhibited by the dimensions and presence of the proximal anchor 700, which will be described in detail below. In the spinal column, distal migration is particularly disadvantageous because the distal anchor 234 may harm the tissue, nerves, blood vessels and/or spinal cord which lie within and/or surround the spine. Such features also reduce the tendency of the distal anchor to cut into the bone during the “window-wiper effect” that is caused by cyclic loading of the device as will be described. In other embodiments, the distal end 232 and/or the outer edges of the helical flange 272 may be sharp and/or configured such that the distal anchor 234 is self tapping and/or self drilling.

A variety of other embodiments for the distal anchor 234 can also be used. For example, the various distal anchors described in U.S. Pat. Nos. 6,887,243 and 6,908,465, which are hereby incorporated by referenced herein. In particular, the distal anchor 234 may comprise a single helical thread surrounding a lumen, much as in a conventional corkscrew. Alternatively, a double helical thread may be utilized, with the distal end of the first thread rotationally offset from the distal end of the second thread. The use of a double helical thread can enable a greater axial travel for a given degree of rotation and greater retention force than a corresponding single helical thread. Specific distal anchor designs can be optimized for the intended use, taking into account desired performance characteristics, the integrity of the distal bone, and whether the distal anchor is intended to engage exclusively cancellous bone or will also engage cortical bone. In still other embodiments, the distal anchor 234 may be formed without a helical flange.

In some embodiments, the device 212 can comprise a proximal anchor 700 and an optional flange 250. The flange 250 can rotate and/or pivot with respect to the proximal anchor 700. In this manner, the bone contacting surface can be positioned more closely to the outer surface of the vertebra. This positioning can result in more bone contacting surface being utilized and the stress supported by the fixation device is spread out over a larger area of the vertebra. However, it should be appreciated that the flange 250 can be omitted from certain embodiments of the fixation device 212.

Another advantage of the illustrated embodiment is that the proximal anchor 700 can be advanced distally over the body 228 while proximal movement of the proximal anchor 700 over the body 228 is resisted. This arrangement allows the clinician to adjust the size (e.g., length) and/or compression force during the procedure without adjusting the position of a distal anchor 234 at the distal end 232 of the body 228. In this manner, the clinician can focus on positioning the distal anchor 234 sufficiently within the vertebra to avoid or reduce the potential for distal migration out of the vertebra, which may damage the particularly delicate tissue, blood vessels, nerves and/or spinal cord surrounding or within the spinal column.

In other embodiments, the proximal anchor 700 can be fixed, coupled and/or integrally formed with the body 228 (e.g., a fixation device in the form of traditional screw or pedicle screw). Various embodiments and/or additional or alternative components of the device 212 can be found in U.S. Patent Publication 2004/0127906 (U.S. patent application Ser. No. 10/623,193, filed Jul. 18, 2003) entitled “METHOD AND APPARATUS FOR SPINAL FUSION”, which is hereby incorporated by reference. Additional embodiments and/or alternative components of the device 212 can be found in U.S. Pat. Nos. 6,951,561, 6,942,668, 6,908,465, and 6,890,333, which are also incorporated by reference.

In some embodiments, the body 228 comprises titanium. However, as will be described in more detail below, other metals, or bioabsorbable or nonabsorbable polymeric materials may be utilized, depending upon the dimensions and desired structural integrity of the finished stabilization device 12 (FIG. 1).

As shown in FIG. 3C, the body 228 is preferably cannulated forming a central lumen 242 to accommodate installation over a placement wire as is understood in the art. The cross section of the illustrated central lumen is circular but in other embodiments may be non circular, e.g., hexagonal, to accommodate a corresponding male tool for installation or removal of the body 228 as explained below. In other embodiments, the body 228 may partially or wholly solid.

With continued reference to FIGS. 3A-C, the proximal end 230 of the body 228 can be provided with a pull pin 238 utilized in compressing the fixation device 212. The pull pin 238 can include a coupling 270, for allowing the body 228 to be coupled to an insertion instrument as described below.

Preferably, the clinician will have access to an array of fixation devices 212, having, for example, different diameters, axial lengths and, if applicable, angular relationships. These may be packaged one or more per package in sterile or non-sterile envelopes or peelable pouches, or in dispensing cartridges which may each hold a plurality of devices 212. The clinician will assess the dimensions and load requirements, and select a fixation device from the array, which meets the desired specifications.

### Methods of Implanting

Methods for implanting stabilization devices described above as part of a particularly advantageous spinal fixation procedure will now be described. Although certain aspects and features of the methods and instruments described herein can be utilized in an open surgical procedure, the disclosed methods and instruments are optimized in the context of a percutaneous or minimally invasive approach in which the procedure is done through one or more percutaneous small openings. Thus, the method steps which follow and those disclosed are intended for use in a trans-tissue approach. However, to simplify the illustrations, the soft tissue adjacent the treatment site have not been illustrated in the drawings.

In some embodiments of use, a patient with a spinal instability is identified. The patient can preferably be positioned face down on an operating table, placing the cervical spinal column into a normal or flexed position as shown in FIG. 1. In some embodiments, the patient can be placed in the prone position on a spinal frame or padded chest bolsters. In some embodiments, the patient can be positioned on a table, such as a radiolucent operating room table, in the surgical position determined to be optimal by the surgeon. General and/or regional anesthesia can be used. The surgical area can then be prepped and draped using sterile techniques.

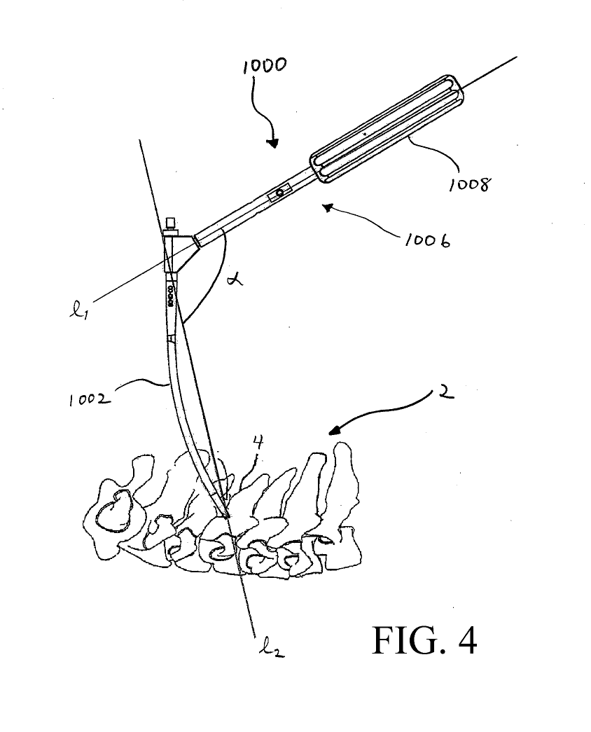

With reference to FIG. 4, a wire introducer 1000 can be inserted through a tissue tract and advanced towards a first vertebra 4 in the cervical spine 2. Depending on surgeon preference, a midline incision can be made over the entry point, or two bilateral incisions slightly off midline may be made. The entry point is the location on the first vertebra 4 at which the wire introducer 1000 is to be positioned. The wire introducer 1000 can be utilized to find the entry point through the stab incision. In some embodiments, fluoroscopy images can be utilized to determine the correct location.

In preferred embodiments, fluoroscopic images can be utilized to best identify the entry point landmarks. The Cephalad/Caudal (Sagittal) entry point is located at the center of the inferior articular process of the superior vertebral body at the treated level. The Medial/Lateral (Coronal) entry point is at the center of the inferior articular process of the superior vertebral body at the treated level.

Once the entry point position is located, the proximal end of the wire introducer 1000 can be moved approximately 5-10 degrees medially to obtain the ideal right to left angulation, or medial trajectory, which can be directed towards the posterior tubercle of the transverse process, or lateral to the foramen transversarium (foramen for the vertebral artery). The medial trajectory can be adjusted to center the spinous process of the level below between the pedicle shadows in the posterior view. The cephalad/caudal angulation can be adjusted to coincide with the lordotic curve of the cervical spine. Upon determining the medial trajectory, the cephalad/caudal angulation, or lateral trajectory is then decided. The initial lateral trajectory can be anterior-caudal, perpendicular to the facet joint, towards the posterior tubercle of the transverse process, and up to the cortical wall-of the superior articular process.

Once the entry point and trajectory have been determined, the wire introducer 1000 can be inserted up to the bone along the determined trajectory. In some embodiments, the wire introducer 1000 can be backed off and repositioned to insure that the trajectory will enter at the appropriate anatomical location. As mentioned previously, in some embodiments several fluoroscopy images can be taken during the positioning process. The wire introducer 1000 can be tapped or seated into the bone so that the entry point is maintained.

As mentioned above, due to the anatomy of the cervical spine 2, the fixation device may need to extend along an axis that when extended interferes with the back of the patient's head (see e.g., FIG. 1). Accordingly, in the illustrated embodiment, the wire introducer 1000 (which will be described in more detail below) includes a cannula portion 1002 and a handle 1006 coupled to the cannula portion 1002. In this manner, a gripping portion 1008 of the handle 1006 is positioned above the cannula portion 1002. This allows the surgeon to grip and securely hold the wire introducer 1000 with reduced interference from the back of the patient's head. Thus, using visualization techniques, the distal end of the trocar 1004 can be advanced towards point toward the vertebra 4 without interfering with the back of the patient's head.

To further compensate for the interference with the patient's head, in some embodiments, the cannula portion 1002 can comprise at least a portion that is curved, as illustrated in FIG. 4. The curved tubular member defines a longitudinal axis, l2 extending generally between the ends of the wire cannula portion 1002. The curved configuration facilitates placing instruments into the wire cannula portion 1002 by allowing the instruments to be inserted into the wire cannula portion 1002 at an angle that is transverse to the longitudinal axis l2 of the curved member. In this manner, a gripping portion 1008 of the handle 1006 is positioned offset from the cannula portion 1002 and interference with the patient's head can be reduced. The handle 1006 has a longitudinal axis l1. The handle 1006 and the cannula portion 1002 can be arranged such that their longitudinal axes l2, l1 form an angle α.

In some embodiments, the wire introducer 1000 can include a trocar 1004 positioned within the cannulated section to help to secure the wire introducer 1000 to the vertebrae. The trocar 1004 can be made of a generally flexible material that can conform to the curved shape of the wire cannula portion 1002. For example, the trocar 1004 can be made of wound wires, spring steel, composites, or other strong and flexible material.

In some embodiments of the wire introducer 1000, the angle α. between the handle 1006 and the cannula portion 1002 is greater than 90 degrees and, in other embodiments, within a range between about 30 degrees and about 150 degrees. In the illustrated embodiment, the angle α. is about 120 degrees. An advantage of the illustrated embodiment is that the surgeon's hand can be positioned offset from the longitudinal axis l2 of the cannula portion 1002. This improves the leverage and ergonomics involved with advancing the wire introducer 1000 through the tissue tract towards the first vertebra 4 in the cervical spine 2.

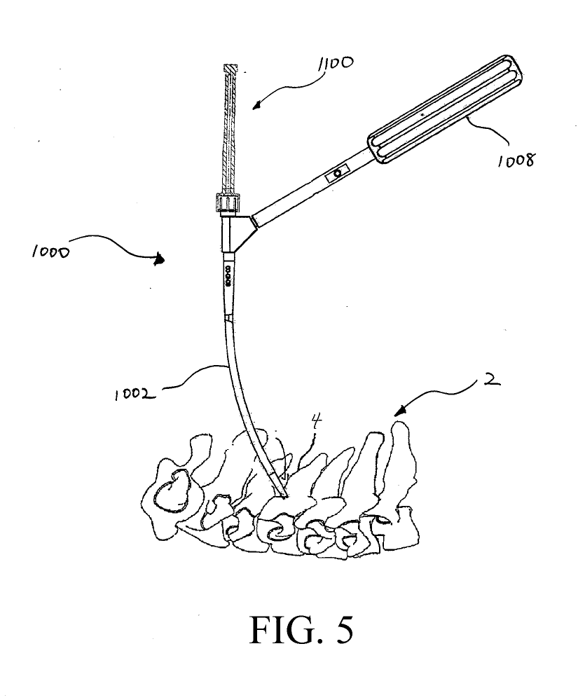

With reference now to FIG. 5, when the end of the wire introducer 1000 is positioned at the desired location on the vertebra 4 and the trajectories have been determined, a strike pin 1100 can be coupled to the proximal end of the introducer 1000. As will be explained in more detail below, mating threads or other coupling features can be provided between the introducer 1000 and the strike pin 1100. The strike pin 1100 can then be tapped with a mallet or hammer (not shown) by the clinician to set the end of the wire introducer 1000 into the facet of the vertebra 4. In some embodiments, a series of lateral fluoroscopy images can be utilized to determine the correct trajectory and/or to ensure that the needle does not compromise the nerve root or the spinal canal. Once the wire introducer 1000 is seated, the strike pin 1100 can be removed by pulling the strike pin 1100 in the proximal direction. In some embodiments, the strike pin 1100 can form part of the wire introducer 1000 and/or the wire introducer 1000 can be lengthened in the proximal direction such that the patient is not contacted when a hammer is used. In another embodiment, the hammer can be used directly against the proximal end of the introducer 1000.

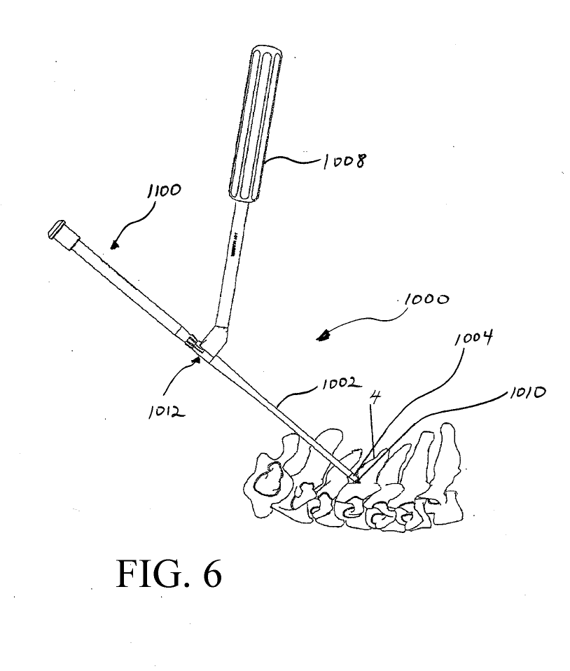

With reference now to FIG. 6, in an alternative embodiment, a trocar 1004 can be used with the wire introducer 1000′ and the end of a trocar 1004 can be positioned at the desired location on the vertebra 4. A strike pin 1100′ can be coupled to the proximal end of the trocar 1004. As will be explained in more detail below, mating threads or other coupling features can be provided between the trocar 1004 and the strike pin 1100′. The strike pin 1100′ can then be tapped with a mallet or hammer (not shown) by the clinician to set the end of the trocar 1004 into the facet of the vertebra 4. This advantageously also sets the sharp distal end 1010′ of the wire introducer 1000′ into the facet. In some embodiments, a series of lateral fluoroscopy images can be utilized to determine the correct trajectory and/or to ensure that the needle does not compromise the nerve root or the spinal canal. Once the wire introducer 1000′ is seated, the strike pin 1100′ can be removed by rotating clockwise. In some embodiments, the strike pin 1100′ can form part of the trocar 1004.

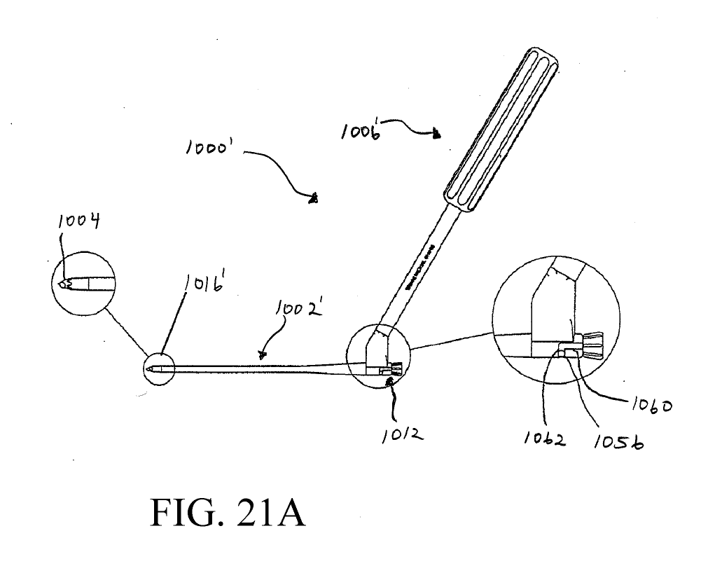

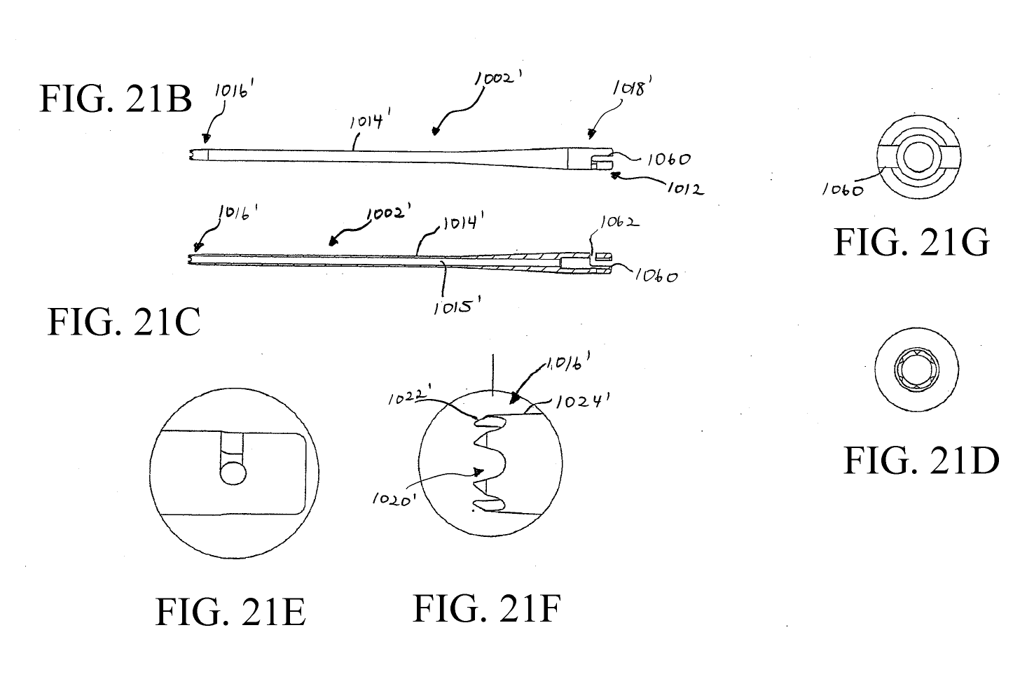

In some embodiments, the trocar 1004 can be removed from the wire introducer 1000. As will be explained in more detail below with respect to FIG. 21A, in the illustrated embodiment, a bayonet connection 1012 can be provided between the introducer 1000 and the trocar 1004. By releasing the bayonet connection 1012, the trocar 1004 can be released and removed from the introducer 1002.

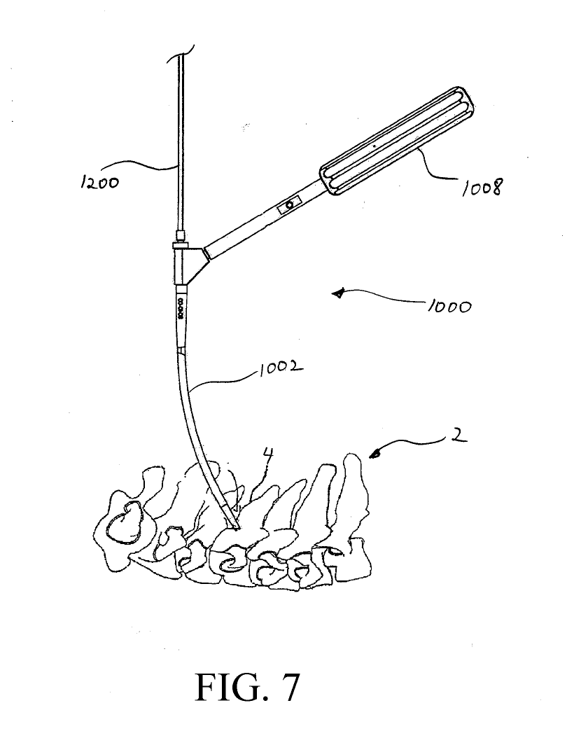

With the trocar 1004 removed, a guidewire drill (e.g., a 0.070 diameter K-wire drill) 1200 can be used as a predrill for the fixation device, as illustrated in FIG. 7. In some embodiments, a drill with a drill bit can be used and can be advanced through the introducer 1000 to the desired fixation device location. In some embodiments, a guidewire having a drill-type distal end can be used. In some embodiments, the trocar 1004 can have a drill-type distal end that can be used to advance the trocar 1004 through the articular processes after tapping the trocar 1004 into the facet of the vertebra 4. The guidewire drill 1200 can then be coupled to a drill (not shown) and then advanced into the vertebra 4 to provide a pre-drill hole for the fixation device.

In preferred embodiments, the guidewire drill 1200 is generally flexible laterally so that it can bend and be advanced through the curved cannula portion 1002, yet generally rigid about its longitudinal axis such that it is able to transfer rotational torque from a drill at a proximal end of the guidewire drill to the drill bit at the distal end of the guidewire drill. In some embodiments, the guidewire drill 1200 can be made of wound wires, spring steel, composites, or other strong and flexible material. Preferably, the guidewire drill is not advanced beyond the distal cortical wall of the superior articular process.

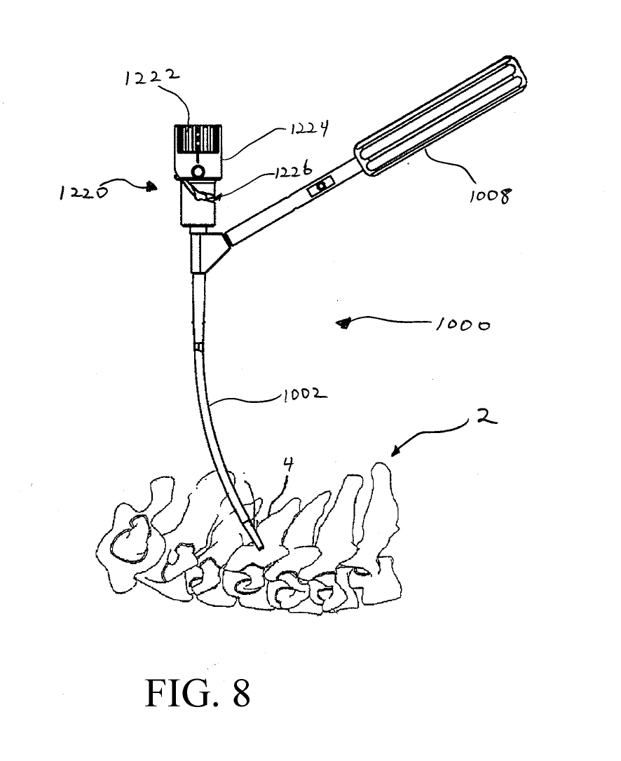

In some embodiments, the wire introducer 1000 can have a drill stop 1220 attached to the proximal end of the cannula portion 1002, as illustrated in FIG. 8. The drill stop 1220 can be welded to the wire introducer 1000, or attached by a plurality of different means, such as adhesives, threaded fasteners, compression fit, etc. The drilling depth for the guidewire drill 1200 can be predetermined and adjusted by changing the length of the drill stop 1220. With reference to FIG. 8, the drill stop 1220 can have a knob 1222 and a housing 1224. The housing 1224 can have a slot 1226 that extends diagonally across the length of the housing 1224. The knob 1222 can have a pin 1228 that is disposed in the slot 1226. When the knob 1222 is rotated, the pin 1228 moves along the slot 1226 to adjust the length of the drill stop. In some embodiments, the drill stop 1220 can be used with the cortex drill 1500 described below. In some embodiments, the length of the drill stop 1220 can be adjusted from at least approximately 12 mm. and/or less than or equal to approximately 16 mm.

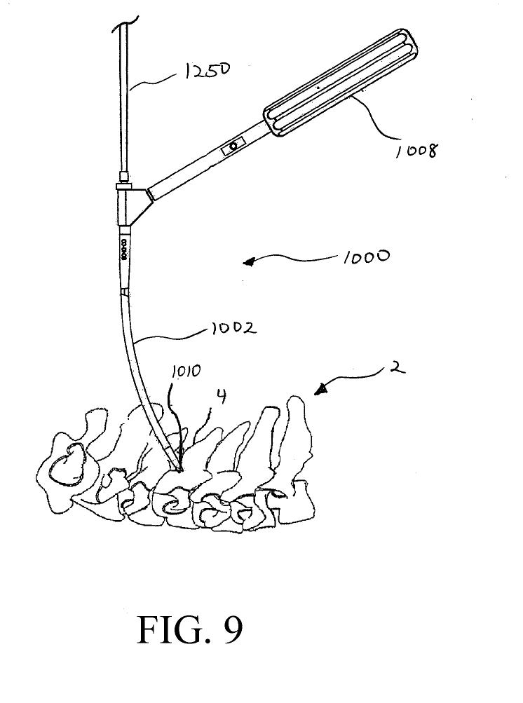

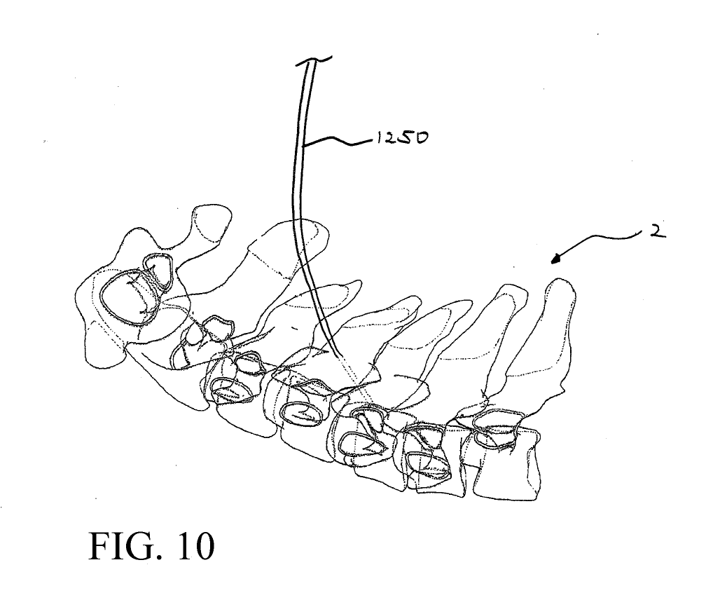

Once the appropriate drilled hole has been completed, the guidewire drill 1200 can be removed and a guidewire 1250 (e.g., a 0.45″ diameter NiTi wire) can be placed through the wire introducer 1000 into the hole, as illustrated in FIG. 9. Advantageously, the guidewire 1250 does not advance through the vertebrae in to the nerves and tissue of the spinal column. Preferably, the distal end of the guidewire 1250 is blunt so that the guidewire 1250 does not inadvertently continue to advance into the articular processes. In some embodiments, the distal end of the guidewire 1250 can have a ball or spherical shape at the distal blunt end. The wire introducer 1000 can then be removed leaving the guidewire 1250 in place, as illustrated in FIG. 10.

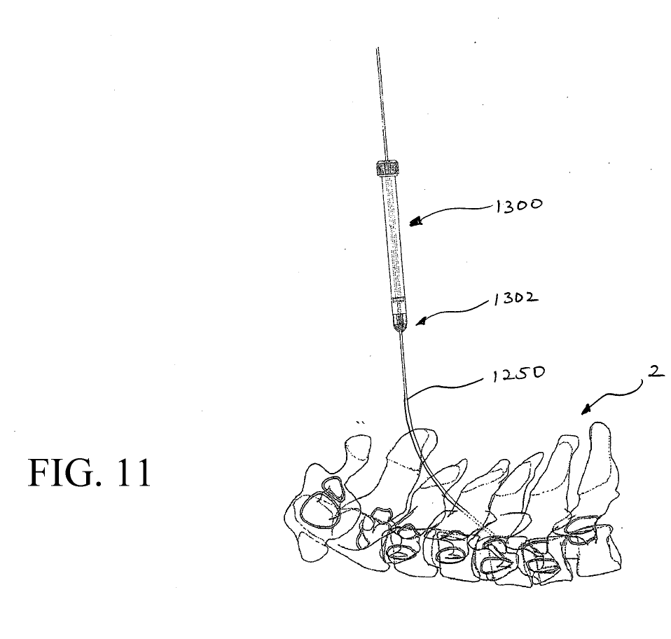

With reference now to FIG. 11, adjacent the guidewire 1250 a small incision (e.g., 8-10 mm length) can be made to accommodate a fascia cutter 1300, which will be described in more detail below. The fascia cutter 1300 can include a sharp distal end 1302 that is configured to cut the tough fascia tissue that lies above the cervical spine. As shown in FIG. 11, the fascia cutter 1300 can be advanced over the guidewire 1250 into the incision. The fascia cutter 1300 can be advanced over the guidewire 1250 until the fascia is sufficiently cut. The fascia cutter 1300 can then be removed leaving the guidewire 1250 in place. Some embodiments of a method to implant a spinal fixation device do not include using a fascia cutter. In some embodiments, cutting the fascia can include cutting with a scalpel in place of or in addition to the fascia cutter 1300.

### Teleport

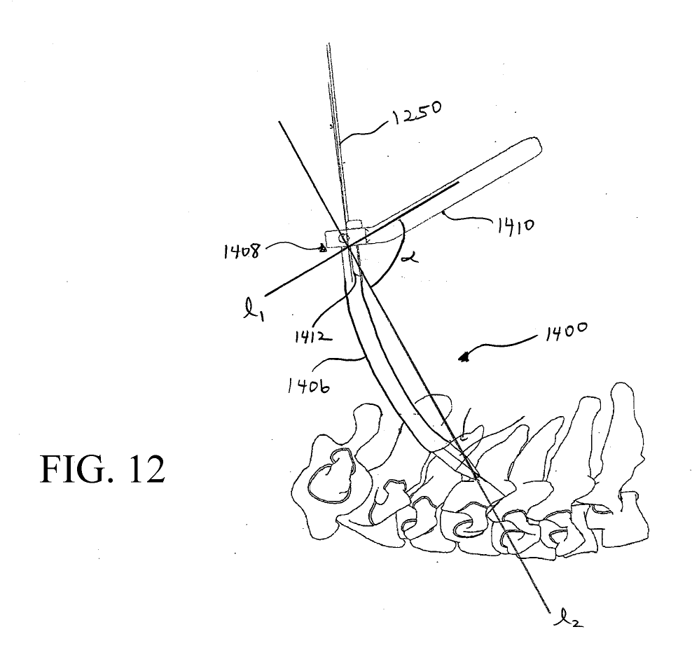

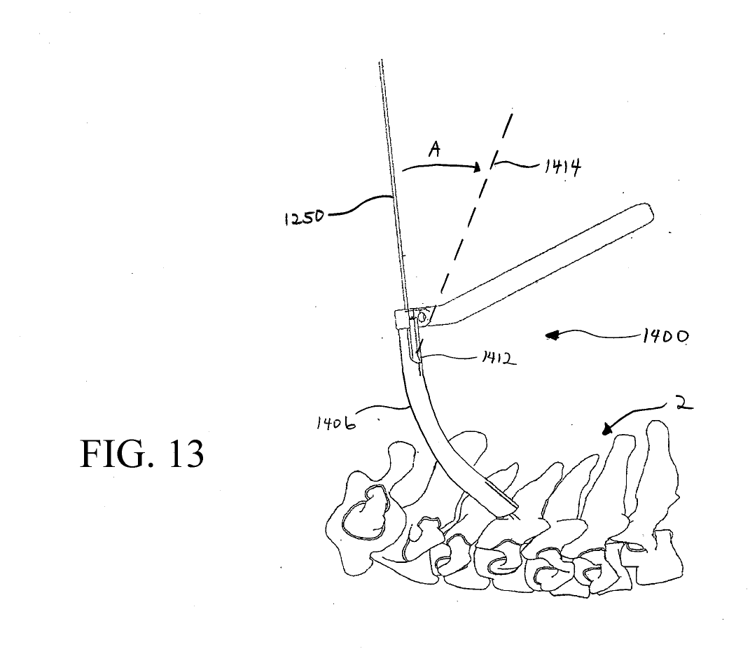

As shown in FIG. 12, a sheath assembly 1400 can be advanced over the guidewire 1250 through the opening until its distal end 1402 reaches the bone in order to retract the tissue to the implant site. In some embodiments, the sheath assembly 1400 can have sheaths that are curved, similar to the curvature of the wire introducer 1000. An embodiment of the sheath assembly 1400 will be described in more detail below. In general, the sheath assembly 1400 is configured to be inserted over the guidewire in a first, low profile, configuration. The sheath assembly 1400 can then be converted to a second, larger profile, configuration, such as illustrated in FIG. 13, in which the sheath assembly 1400 provides a larger access lumen to the target site (e.g., the vertebrae). In the illustrated embodiment, the sheath 1400 includes inner and outer sheaths 1404, 1406 in a manner as described in U.S. Patent Publication No. 2006/0030872, filed Aug. 3, 2004, application Ser. No. 10/911,215 which is hereby incorporated by reference herein in its entirety. In the first configuration (FIG. 12), the sheath assembly 1400 can be advanced until the distal end 1402 of the inner sheath 1404 reaches the bone. An actuator 1408 can then be released to advance the outer sheath 1406 downward over the inner sheath 1404 until the outer sheath 1406 is resting on the facet (see FIG. 13). The inner sheath 1404 can be removed, preferably leaving the guidewire 1250 and outer sheath 1406 in place. In some embodiments, the tip 1402 of the inner sheath 1404 and/or the tip of the outer sheath 1406 can be barbed or spiked to secure the sheaths 1404, 1406 against the vertebrae. Preferably, the outer sheath 1406 has an inner diameter that is at least approximately 7 millimeters in diameter. In some embodiments, the inner diameter of the outer sheath 1406 can be at least approximately 5 millimeters and/or less than or equal to approximately 20 millimeters.

As mentioned above, due to the anatomy of the cervical spine 2, the fixation device may need to extend along an axis that, when extended, interferes with the back of the patient's head (see e.g., FIG. 1). Accordingly, as shown in FIGS. 12 and 13, the sheath assembly 1400 can include a handle 1410 that is coupled at a transverse angle to the outer sheath 1406. Furthermore, in some embodiments, the inner sheath 1404 and outer sheath 1406 can be curved members. The curved tubular members define a longitudinal axis, l2 extending generally between the ends of the sheaths. The curved configuration facilitates placing instruments into the outer sheath 1406 by allowing the instruments to be inserted into the sheaths at an angle that is transverse to the longitudinal axis l2 of the outer sheath 1406. In this manner, interference with the patient's head can be reduced.

The handle 1410 has a longitudinal axis l1. Similar to the handle 1008 of the wire introducer 1000, the handle 1410 and the outer sheath 1406 can be arranged such that their longitudinal axes l1, l2 form an angle α. In this manner, the handle 1410 can be positioned offset from the outer sheath 1406. This offset positioning allows the surgeon to grip and securely hold the outer sheath 1406 with reduced interference from the back of the patient's head.

In some embodiments, the angle α between the handle 1410 and the outer sheath 1406 is greater than 90 degrees and, in other embodiments, within a range between about 30 and about 150 degrees. In the illustrated embodiment, the angle α. is about 120 degrees. An advantage of the illustrated embodiment is that the surgeon's hand can be positioned offset from the longitudinal axis l2 of the outer sheath 1406. This offset positioning improves the leverage and ergonomics involved with holding the outer sheath 1406 in place during the various procedures described below.

The outer sheath 1406 can desirably also include an elongated proximal opening or slot 1412, which generally faces the handle 1410. With reference to FIG. 13, the slot 1412 facilitates placing instruments into the outer sheath 1406 by allowing the instrument to be moved in the direction A towards line 1414, which is transverse to the longitudinal axis 12 of the outer sheath 1406. In this manner, interference with the patient's head can be reduced.

In some embodiments, the guidewire 1250 can be removed after placement of the sheath assembly 1400, since the outer sheath 1406 can provide an access path to guide instruments to the implant site. As mentioned above, barbed or spiked tips of the inner sheath 1404 and/or outer sheath 1406 can help secure the 1404, 1406 against the vertebrae. In some embodiments, the guidewire 1250 can remain coupled to the articular processes and the instruments inserted through the outer sheath 1406 can be cannulated. In the subsequent descriptions, the embodiments will be described with the guidewire remaining attached to the articular processes.

In some embodiments, tools to prepare the facets for implanting the fixation device can be delivered through the outer sheath 1406. For example, a rasping tool can be inserted through the outer sheath 1406 to roughen the facets and enhance osseointegration. Preferably, the elongate member on which the rasping device is attached is flexible so that the tool can be advanced through the curvature of the outer sheath 1406. Yet, the elongate member can be somewhat rigid so that it can transmit axial forces for the rasping process. In some embodiments, other tools and devices can be delivered through the outer sheath 1406 to the implant site.

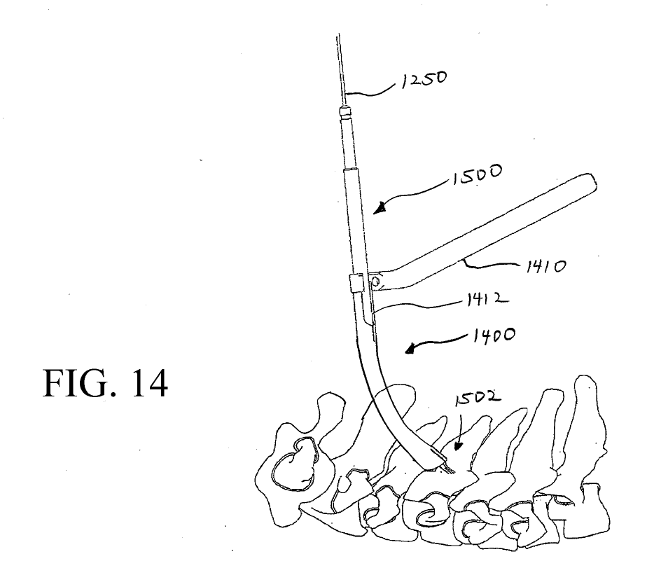

With reference now to FIG. 14, a cortex drill 1500 can be advanced towards the vertebrae through the sheath assembly 1400 and over the guidewire 1250. In some embodiments, the cortex drill 1500 can be cannulated through its longitudinal length to receive the guidewire 1250. As will be explained in more detail below, the cortex drill 1500 preferably can be powered to make a clearance hole for the implant and counter sink in the facet for the proximal anchor. In some embodiments, the cortex drill 1500 preferably includes a flexible elongated transmission member as will be described below. This flexible transmission member can allow the cortex drill 1500 to be advanced through the curvature of the outer sheath 1406. Furthermore, this flexible transmission member allows a proximal end of the cortex drill 1500 to be flexed in the direction of arrow A and line 1414 of FIG. 13 while a distal end 1502 of the cortex drill 1500 maintains a desired position and orientation with respect to the vertebrae. As will be explained below, the distal end 1502 of the cortex drill 1500 can be configured to form a clearance hole and/or counter sink for the fixation device to be inserted into the vertebrae. In some embodiments, the cortex drill 1500 can be coupled to a power instrument.

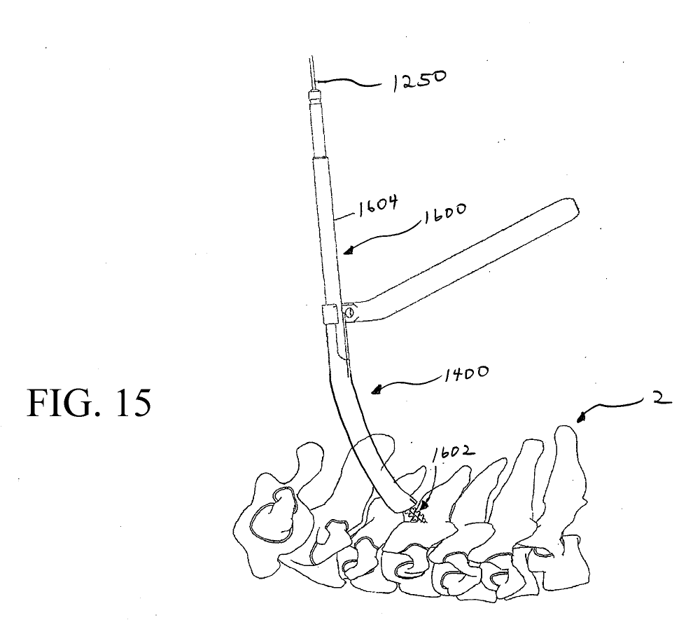

After the cortex drill 1500 is removed, a tapping device 1600 can be advanced over the guidewire 1250, as illustrated in FIG. 15. In some embodiments, the tapping device 1600 can be cannulated through its longitudinal length to receive the guidewire 1250. In some embodiments, the tapping device 1600 is rotated, by hand, and advanced into the vertebrae. Preferably, the depth of the tapping device 1600 is verified using fluoroscopy. As will be explained in more detail below, the tapping device 1600 preferably includes a handle (not shown in FIG. 15) at a proximal end and a tapping portion at a distal end 1602. The handle and distal end 1602 can desirably be connected by a body 1604 that can be a flexible rotation transmission member. In some embodiments, the handle can be connected to the body 1604 by a quick connector. The flexible body 1604 can allow the tapping device 1600 to be advanced through the curvature of the outer sheath 1406. In other embodiments of the device, the fixation implant can be configured to be self-tapping. In such an embodiment, the tapping device 1600 can be eliminated.

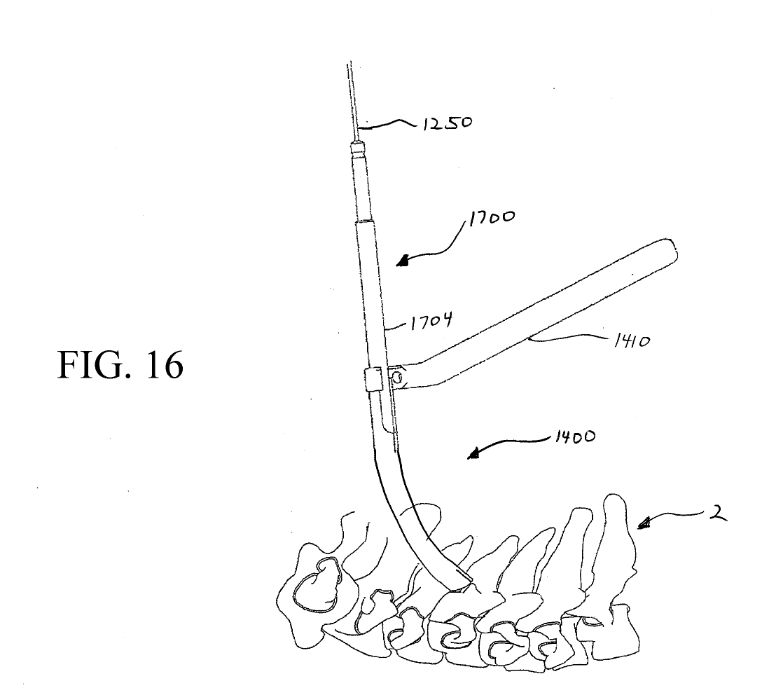

With a hole tapped, the tapping device 1600 can be removed from the sheath assembly 1400. Then, with reference to FIG. 16, a fixation device (e.g., the fixation device 212 as described above) can be loaded onto a driver 1700. In some embodiments, the driver 1700 can be cannulated through its longitudinal length to receive the guidewire 1250. Then, the driver 1700 can be used to advance a fixation device over the guidewire 1250, through the sheath assembly 1400 to the vertebrae. Preferably, the depth of the fixation device is verified using fluoroscopy. In some embodiments, the fixation device can be implanted such that the proximal end protrudes from the vertebrae so that subsequent compression of the fixation device can be accomplished.

As will be explained below, the distal end 1702 (not shown in FIG. 16) of the driver 1700 is configured to removably engage a proximal end of the fixation device. After the proper position of the fixation device has been established, in some embodiments, the driver 1700 can be removed by pulling it off of the fixation device. In some embodiments, the driver 1700 can be removed by rotating the driver 1700 to unfasten from the fixation device. The driver 1700 preferably also includes a flexible rotation member 1704 as further described below.

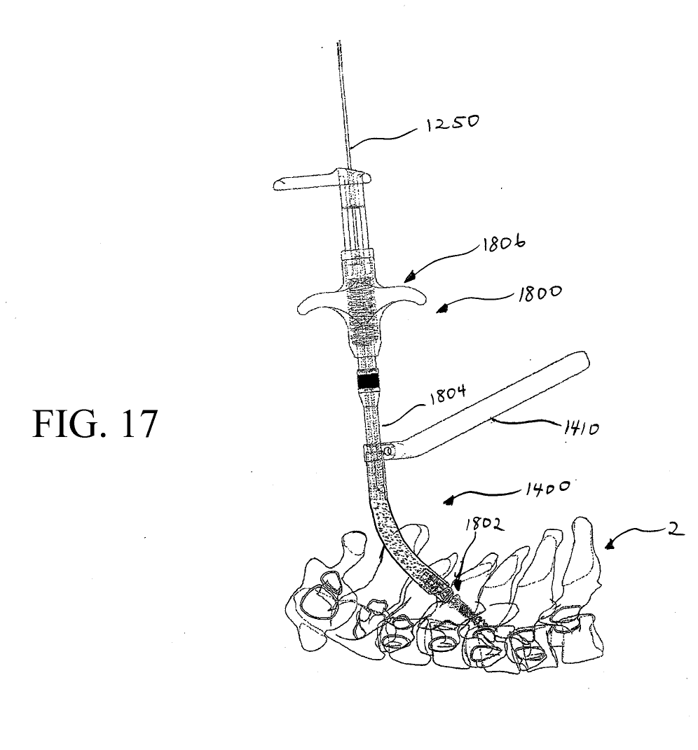

With the distal anchor 234 of a fixation device 212 positioned properly in the vertebrae, the driver 1700 can be decoupled from the fixation device and removed from the sheath assembly 1400. A compression device 1800, as illustrated in FIG. 17, which will be described in more detail below, can then be advanced over the guidewire 1250 and through the sheath assembly 1400. Preferably, the compression device 1800 is cannulated through its longitudinal length to receive the guidewire 1250. As will be explained in detail below, the compression device 1800 can include a distal end 1802, a handle 1806 and flexible transmission member 1804 extending between the distal end 1802 and handle 1806. The distal end 1802 can be configured to engage the coupling 270 on the pull pin 238 of the fixation device 212. The flexible transmission member 1804 can allow the compression device 1800 to be advanced through the curvature of the sheath assembly 1400.

The compression device 1800 can be used to advance the proximal anchor 700 over the body 228 of the fixation device 212. Once the distal end 1802 of the compression device 1800 is attached to the coupling 270 on the pull pin 238 of the fixation device 212, the handle 1806 can be squeezed to advance the proximal anchor 700 and apply compression to the fixation device 212. Lateral fluoroscopy can be used to confirm compression of the fixation device 212. Once compression has been confirmed, the handle 1806 can be released and the compression device 1800 removed.

In this manner, the proximal anchor 700 can be advanced distally with respect to the body 228 until the proximal anchor 700 fits snugly against the outer surface of the vertebra or a fixation plate/rod. As explained above, one advantage of the structure of the illustrated embodiments is the ability to adjust compression independently of the setting of the distal anchor 234 within the vertebra. That is, with the distal anchor properly positioned within the inferior vertebra, proper compression (and/or length of the device) between the superior and inferior vertebrae is achieved by advancing the proximal anchor over the body (and/or retracting the body with respect to the proximal anchor).

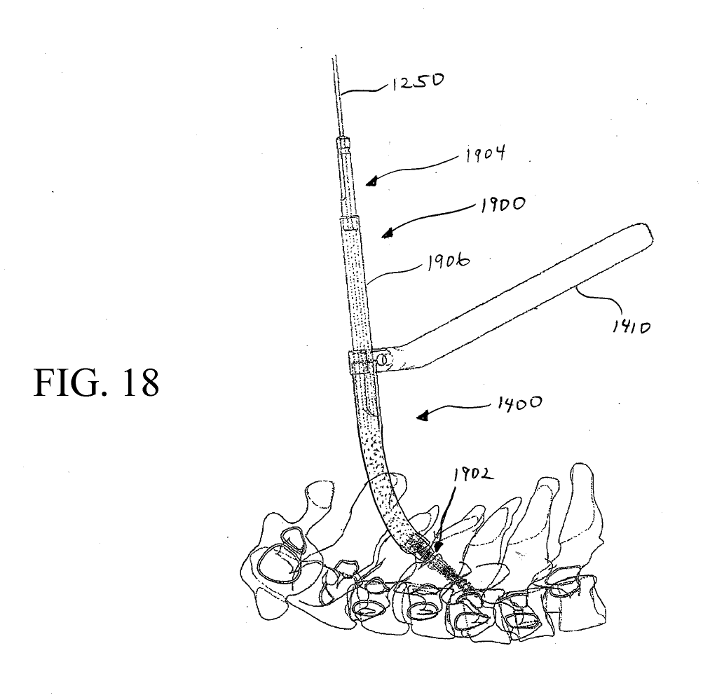

As shown in FIG. 18, the pull pin 238 of the fixation device 212 can then be removed using a pin remover 1900, which will be described in further detail below. As with the tools used with the sheath assembly 1400 described above, the pin remover 1900 can be cannulated through its longitudinal length to receive the guidewire 1250. The pin remover 1900 preferably includes a distal end 1902, a proximal end 1904 and a flexible body 1906 extending therebetween. The distal end 1902 can be configured to couple with the coupling 270 of the pull pin 238. In some embodiments, the distal end 1902 can have a quick connect coupling. The flexible body 1906 can allow the pin remover 1900 to be advanced through the curvature of the sheath assembly 1400. To remove the pull pin 238, in some embodiments, the pin remover 1900 can be rotated, which in turn rotates the pull pin 238 to unscrew it from the body 228 of the fixation device 212. In other embodiments, the pull pin 238 can be attached to the body 228 through means other than threaded connection. In some embodiments, the pull pin 238 can be left in the patient. In other embodiments, the second portion can be partially removed by cutting the pull pin 238.

In some embodiments, a funnel 2000 can be used to deliver substances to the implant site. For example, allograft material can be delivered to help with osseointegration of the fixation device 212 with the vertebrae. To deliver the allograft material, the allograft material can be inserted into the funnel tube 2002 and the funnel 2000 carrying the material can be advanced along the guidewire 1250. A plug 2006 can be placed in the funnel tube 2002 to help prevent the allograft material from escaping as the funnel 2000 is advanced along the guidewire 1250. A rod 2008 can be used to push the allograft material distally out of the funnel 2000 when the implant site is reached. In an alternative method of use, the funnel 2000 can be inserted first along the guidewire 1250 and the allograft material can be pushed to the implant site using the rod 2008 that is inserted through the funnel tube 2002.

After the fixation device 212 is implanted, the sheath assembly 1400 and the guidewire 1250 can be removed. Confirmation of proper fixation device 212 placement and removal of pull pin 238 should be confirmed prior to removing the guidewire 1250. The access site may be closed and dressed in accordance with conventional wound closure techniques and the steps described above may be repeated on the other side of the vertebrae for substantial bilateral symmetry. The bone stabilization devices 212 may be used alone or in combination with other surgical procedures such as laminectomy, discectomy, artificial disc replacement, and/or other applications for relieving pain and/or providing stability.

It should be appreciated that not all of the steps described above are critical to procedure. Accordingly, in some embodiments, some of the described steps may be omitted or performed in an order different from that disclosed. Further, additional steps may be contemplated by those skilled in the art in view of the disclosure herein, without departing from the scope of the present inventions. In addition, while the above-described methods are described with reference to the cervical spine and a trans-facet application, in other embodiments, certain aspects and features of the devices and techniques herein can be used in other portions of the spine (e.g., lumbar) and/or other techniques (e.g., pedicle screws and constructs). They can also be used with other procedures (e.g., anterior cervical decompression and fusion, ACDF).

### Devices

Additional details of the various tools and components described above will now be presented.

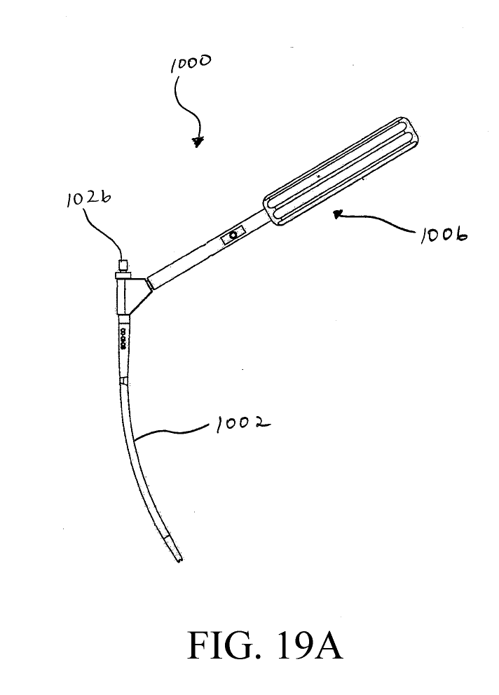

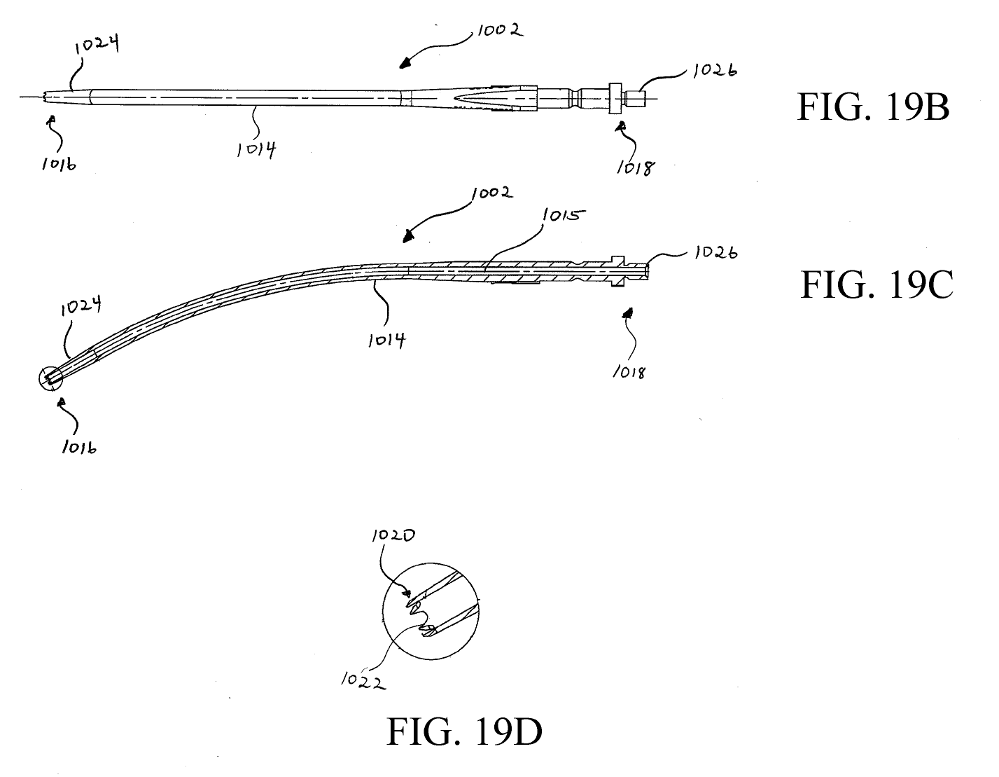

FIGS. 19A-D and 20A-B illustrate various views of an embodiment of the wire introducer 1000 and strike pin 1100. With initial reference to FIG. 19A, the illustrated wire introducer 1000 generally comprises a cannula portion 1002 coupled to a handle 1006. As shown in FIGS. 19A and 19B, the cannula portion 1002 can comprise a generally tubular, elongated curved body 1014 that defines an inner lumen 1015. In some embodiments, the cannula portion 1002 is rigidly curved in a predetermined shape that is suited for accessing the cervical vertebrae while helping avoid interference with the patient's head. The body 1014 includes a distal end 1016, and a proximal end 1018 which can include a connector projection 1026.

With reference to FIG. 19D, in some embodiments, the distal end 1016 can preferably include a plurality of teeth 1020 with sharpened edges 1022. The teeth 1020 and edges 1022 can be configured to aid the insertion of the distal end 1016 of the introducer 1000 through the patient's tissue and in embedding the wire introducer 1000 into the vertebrae. The distal end 1016 preferably has a tapered outer profile 1024 as shown in FIGS. 19B-C.

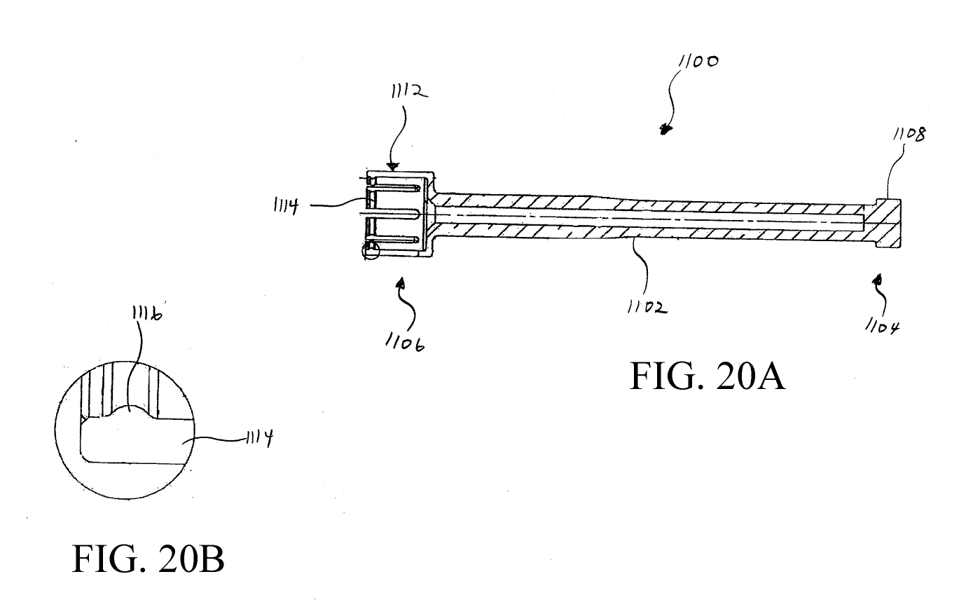

With reference now to FIGS. 20A-B, a strike pin 1100 that can couple with the wire introducer 1000 will now be described in more detail. As mentioned above, the strike pin 1100 can be used to set the tip of the wire introducer 1000 into the facet. In the illustrated embodiment, the strike pin 1100 comprises a generally elongated body 1102 with a proximal end 1104 and a distal end 1106. The proximal end 1104 can include an enlarged portion 1108, which can be configured to receive a striking force from a hammer or mallet. The distal end 1106 of the device can include a connector 1112, which is configured to be coupled with the connector projection 1026 on the wire introducer 1000. In the illustrated embodiment, the connector 1112 includes prongs 1114 that can be placed over the connector projection 1026. The prongs 1114 can include snap protrusions 1116 that slide over the connector projection 1026 to help prevent the strike pin 1100 from inadvertently releasing from the wire introducer 1000. In some embodiments, the strike pin 1100 and wire introducer 1000 can be coupled with other mechanisms, such as, threads, spring detents, O-rings etc.

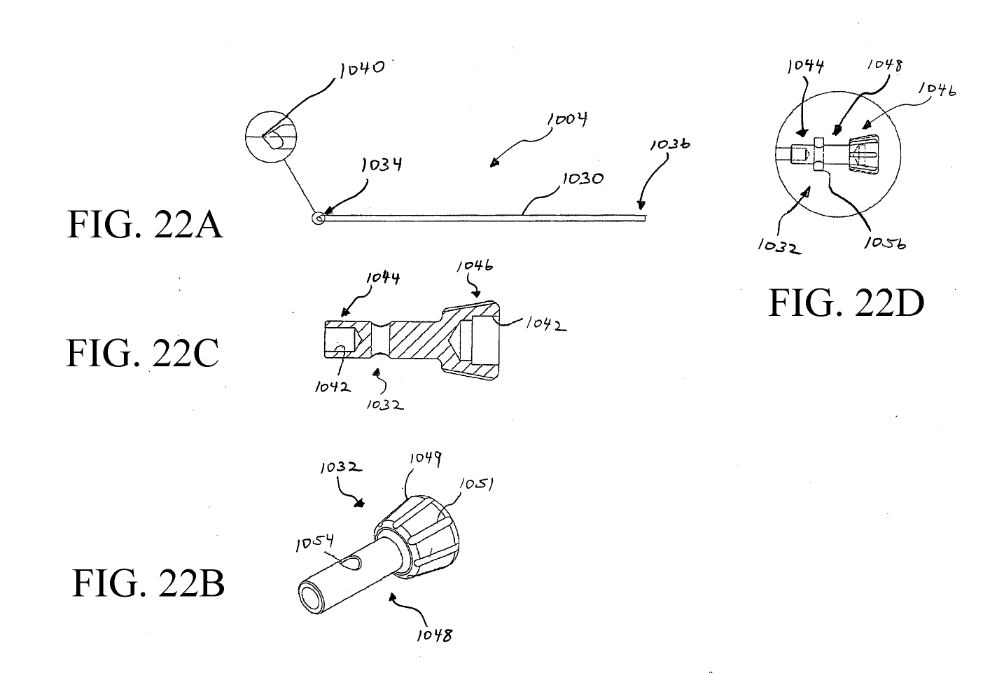

FIGS. 21A-21F and 22A-D illustrate various views of another embodiment of the wire introducer 1000′ and a trocar 1004. With initial reference to FIG. 21A, the illustrated wire introducer 1000′ generally comprises a cannula portion 1002′ coupled to a handle 1006′. As shown in FIGS. 21B and 21C, the cannula portion 1002′ can comprise a generally tubular, elongated curved body 1014′ that defines an inner lumen 1015′, which is configured to receive a curved trocar 1004. In some embodiments, the cannula portion 1002′ is rigidly curved in a predetermined shape that is suited for accessing the cervical vertebrae while helping avoid interference with the patient's head. The body 1014′ includes a distal end 1016′, and a proximal end 1018′ which can include part of the bayonet connection 1012′ mentioned above.

With reference to FIG. 21F, in some embodiments, the distal end 1016′ can preferably include a plurality of teeth 1020′ with sharpened edges 1022′. The teeth 1020′ and edges 1022′ can be configured to aid the insertion of the distal end 1016′ of the introducer 1000′ through the patient's tissue and in embedding the wire introducer 1000′ into the vertebrae. The distal end 1016′ preferably has a tapered outer profile 1024′ as shown in FIG. 21F.

FIG. 22A illustrates a first portion 1030 of the trocar 1004. The first portion 1030 can comprise an elongated body with a distal end 1034 and a proximal end 1036. At least part of the first portion 1030 can be flexible so that it can be advanced through the curvature of the cannula portion 1002′. Preferably, the first portion 1030 is rigid and generally not compressible along its longitudinal length so that it can transmit impact forces when the trocar 1004 is struck with a mallet, as described above. The distal end 1034 preferably includes a sharpened tip 1040, which is configured to pierce tissue. The proximal end 1036 is configured to be coupled to a handle 1032 (or integrally formed therewith), which is shown in FIGS. 22B-D. In the illustrated embodiment, the proximal end 1036 of the first portion 1030 is press fitted into a cavity 1042 formed in the handle 1032.

With reference to FIGS. 22C-D, the handle 1032 preferably includes a distal end 1044, a proximal end 1046 and a middle portion 1048 extending therebetween. The distal portion 1044 includes the cavity 1042 described above. The proximal portion 1046 can include an enlarged diameter gripping portion 1049, which can include gripping features 1051 such that the trocar 1004 can be grasped and rotated. The proximal end can also include a cavity 1050 for receiving a distal end of a strike pin 1100 as will be described below. In some embodiments, the cavity 1050 can include threads (not shown) that are complementary to threads on the proximal end 1036 of the first portion 1030.

As illustrated in FIGS. 22B and 22D, the middle portion 1048 preferably includes a through hole 1054, which extends generally perpendicularly with respect to the longitudinal axis of the trocar 1004. A bayonet pin 1056 can be positioned within the through hole 1054 with its ends protruding beyond the surface of the middle portion 1048.

With reference back to FIG. 21A, when the trocar 1004 is positioned within the wire introducer 1000, the sharpened tip 1040 of the trocar can extend beyond the distal end 1016′ of the wire introducer 1000′. Together the wire introducer 1000′ and trocar 1004 can form a sharpened tip that is configured to pierce tissue. In some embodiments, a stab incision may need to be used to introduce the wire introducer into the patient. In the illustrated embodiments, the wire introducer 1000′ and trocar 1004 are coupled together by the bayonet connection 1012. Specifically, with reference to FIGS. 21A, 21C, 21E, 21G, the proximal end 1018′ of the wire introducer 1000′ includes a slot or groove 1060, which extends along the longitudinal axis of the introducer 1000′. The groove 1060 terminates in a side groove 1062 to form a L-shaped bayonet connection 1012. Thus, the trocar 1004 can be secured within the wire introducer 1000′ when the pin 1056 is positioned within the side groove 1062. To remove the trocar 1004 from the wire introducer, the wire introducer 1000′ can held in place with the handle 1006′ with one hand while the other hand grips the gripping portion 1049 of the trocar 1004 and rotates the trocar 1004 to align the pin 1056 with the groove 1060. The trocar 1004 can then be withdrawn and removed from the wire introducer 1000′.

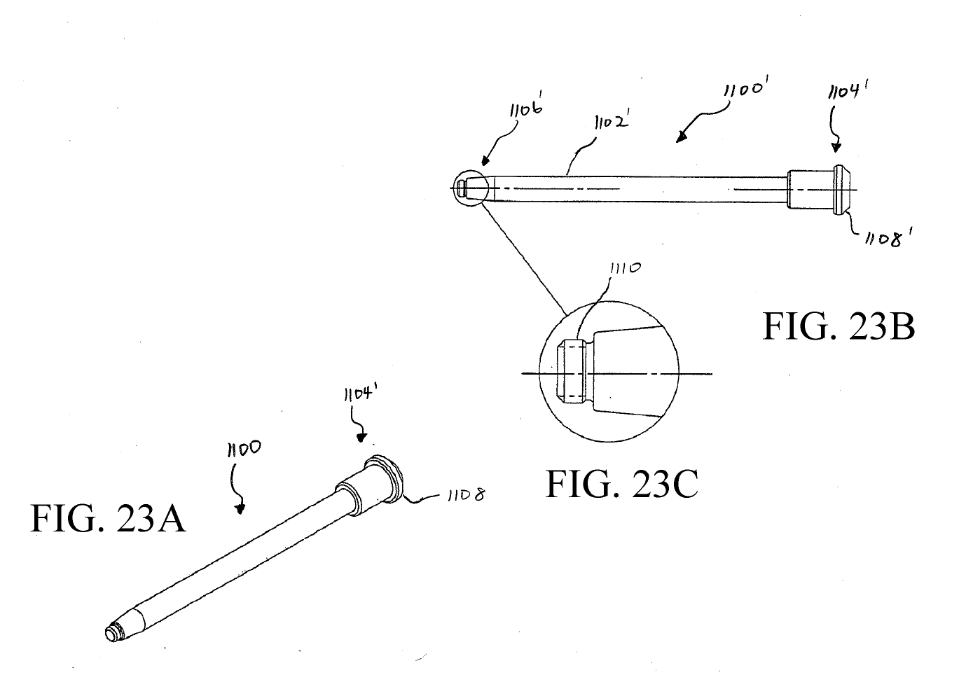

With reference now to FIGS. 23A-C, a strike pin 1100′ that can couple with the trocar 1004 will now be described in more detail. As mentioned above, the strike pin 1100′ can be used to set the tip of the trocar 1004 into the facet. In the illustrated embodiment, the strike pin 1100′ comprises a generally elongated body 1102′ with a proximal end 1104′ and a distal end 1106′. The proximal end 1104′ can include an enlarged portion 1108′, which can be configured to receive a striking force from a hammer or mallet. The distal end 1106′ of the device can included a threaded portion 1110′, which is configured to be threaded into the cavity 1050 of the trocar 1004. In this manner, the strike pin 1100′ can coupled to the wire introducer 1000′ and trocar 1004. In some embodiments, the strike pin 1100′ and cavity 1050 can be formed with other mechanisms for coupling the two components together (e.g., prongs, O-rings etc.). In the embodiments that include threads, the threads are preferably configured such that coupling the strike pin 1100′ to the trocar 1004 involves rotating the strike pin 1100′ in a direction (e.g., clockwise) that is the same direction which is used to rotate the trocar 1004 to release it from the bayonet connection 1012. After the trocar 1004 is set into the facet, the trocar 1004 can be removed from the introducer 1000′ while remaining coupled to the strike pin 1100′ or, in other embodiments, the strike pin 1110′ can be decoupled from the trocar 1004 before the trocar is removed from the introducer 1000′.

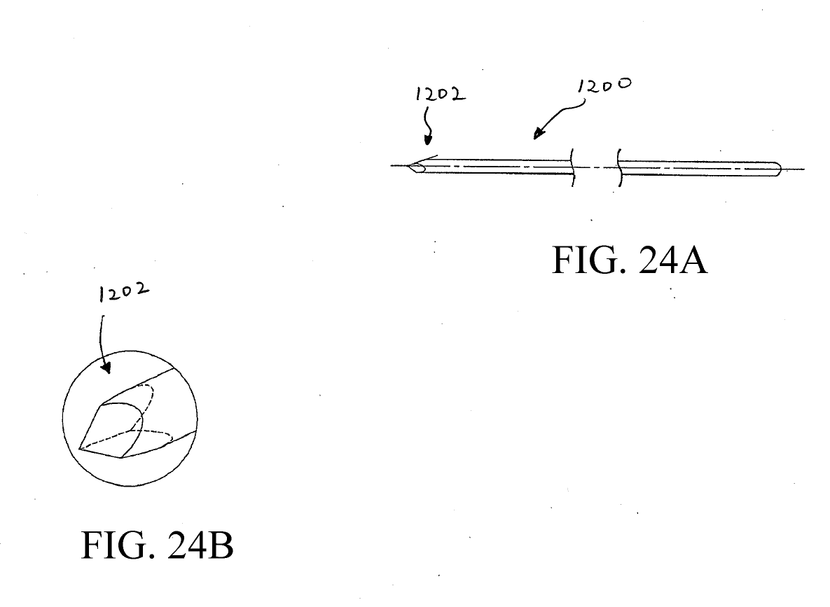

FIGS. 24A-B illustrate the guidewire drill 1200 shown in FIG. 10. As shown, in the illustrated embodiment, the guidewire drill 1200 can include a sharpened or trocar-type tip 1202. In other embodiments, the tip 1202 can have a cutting edge similar to drill bits. As mentioned above, this guidewire drill 1200 can be coupled to a drill with a wire driver to pre-drill a small hole into the vertebrae.

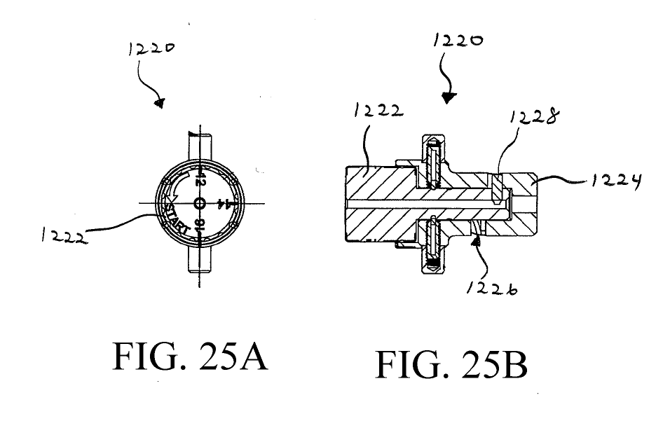

FIGS. 25A-B illustrate an embodiment of a drill stop 1220. The drill stop 1220 includes a knob 1222 rotatably coupled with a housing 1224. As described above, the drilling depth for the guidewire drill 1200 can be predetermined and adjusted by changing the length of the drill stop 1220. The drill stop 1220 can include a knob 1222 and a housing 1224. In the illustrated embodiment, the housing 1224 has a slot 1226 that extends diagonally across the length of the housing 1224. The knob 1222 can have a pin 1228 that is disposed in the slot 1226. When the knob 1222 is rotated, the pin 1228 moves along the slot 1226 to move the knob 1222 and housing 1224 closer together or farther apart in the proximal-distal direction, effectively adjusting the length of the drill stop 1220. In some embodiments, the length of the drill stop 1220 can be adjusted from at least approximately 12 mm. and/or less than or equal to approximately 16 mm. Preferably, the drill stop 1220 is cannulated so that the guidewire 1250 can extend through it.

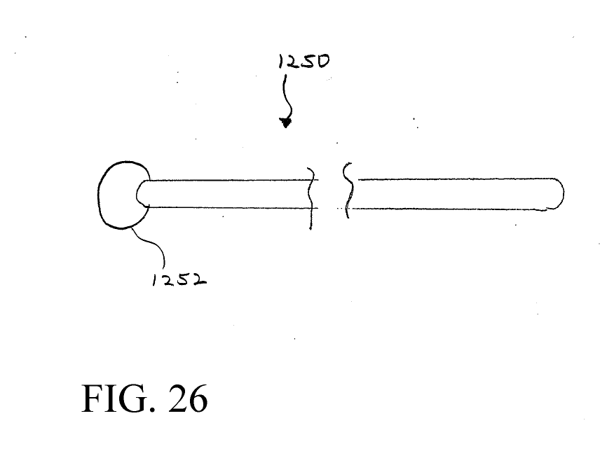

FIG. 26 illustrates the blunt ended guidewire 1250, which is shown previously in FIG. 9. This guidewire 1250 can be inserted into the hole formed by the sharp ended guidewire 1200 described above. The guidewire 1250 can then be used to guide various instruments which are advanced over the guidewire 1250. The sharpened guidewire drill 1200 can be used to form the initial hole and the blunt guidewire 1250 can be used to guide instruments. In some embodiments, the guidewire can have a sphere or ball 1252 attached to an end to prevent the guidewire 1250 from inadvertently advancing into the spinal column, which can cause harm to the patient.

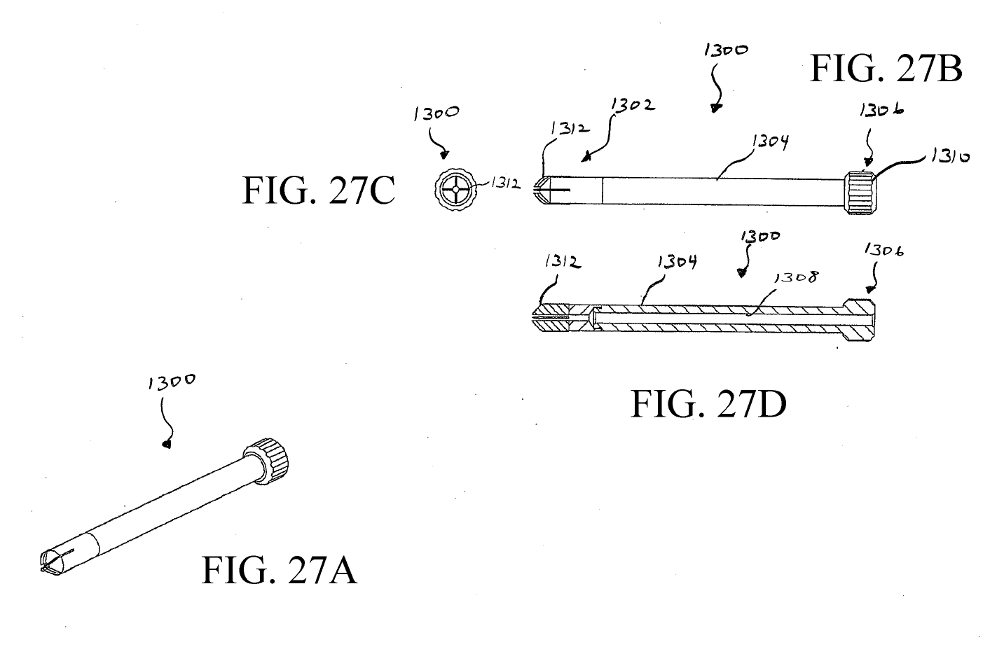

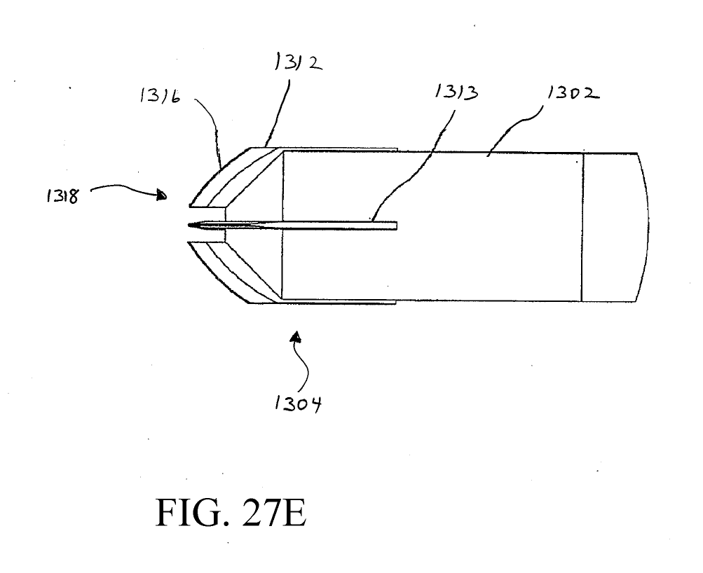

The fascia cutter 1300, which was introduced in FIG. 11, will now be described with reference to FIGS. 27A-E. As shown, in the illustrated embodiment, the fascia cutter 1300 can include a generally elongated flexible body 1304 that has a distal end 1302 and a proximal end 1306. The body 1304 preferably defines a guidewire lumen 1308 such that the cutter 1300 can be advanced over the guidewire 1250 described above. The flexible body 1304 can advantageously follow the curved path of the guidewire 1250.

The proximal end 1306 of the cutter 1300 can include an enlarged diameter portion 1310 with knurling or other gripping features to facilitate manipulation of the cutter 1300. The distal end 1302 of the device preferably includes a plurality of cutting instruments 1312 which are configured to cut the fascia in the cervical region of the patient.

With reference to FIG. 27E, in the illustrated embodiment, the cutter 1300 includes four cutting elements 1312 arranged with slots 1313 formed in the body 1304. In the illustrated embodiment, the cutting elements 1312 are generally equi-angularly positioned about the body 1304 and, thus are arranged at about 90 degrees angular spacing with respect to each other about the body 1304. Each of the cutting elements 1312 preferably includes an accurate shaped cutting edge 1316 that terminates at a distal end in a sharp tip 1318. In other embodiments, other numbers and configurations of cutting elements 1312 can be included on a cutter. An advantage of the illustrated embodiment is that a plurality of cutting elements 1312 can be positioned on the distal end of cutters and each of the plurality of cutting elements can define a cutting edge that extends generally radially from the distal end of the guidewire lumen. Thus, the fascia cutter 1300 can be advanced over the guidewire and used to cut the fascia.

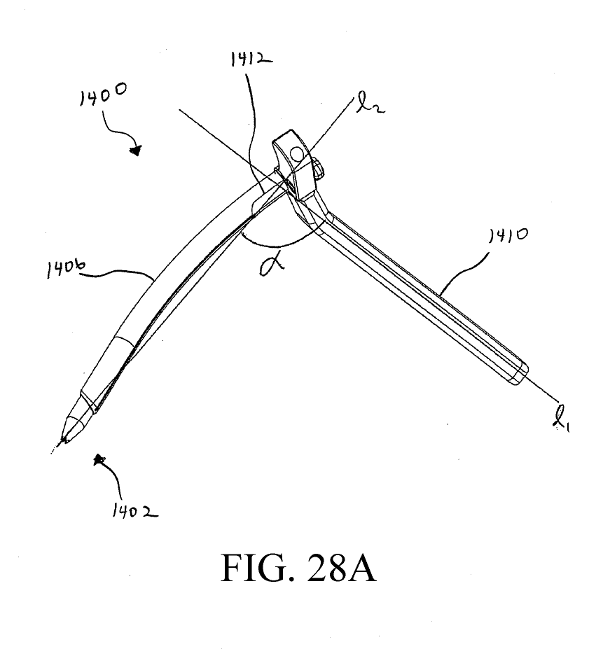

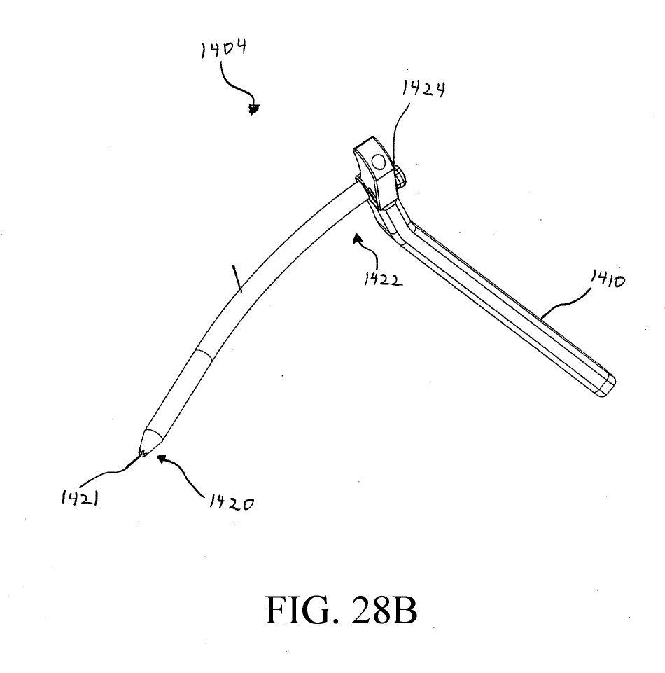

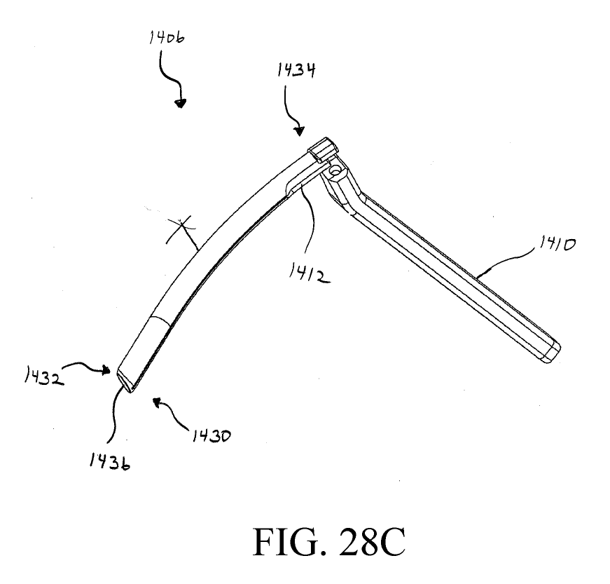

FIGS. 28A-C illustrate in more detail the sheath assembly 1400 introduced above. As shown in FIGS. 28A and 28B, the sheath 1400 can include the first dilator tube or inner sheath 1404 having a distal end 1402 with a tapered tip 1420, and a proximal end 1422 with a locking member 1424, which extends radially from the inner sheath 1404. The inner sheath 1404 can have a curved profile. In some embodiments, the inner sheath 1404 can be flexible such that it can conform to the curved profile of the outer sheath 1406. As illustrated in FIG. 28A, the inner sheath 1404 can define a longitudinal axis, l2 that is transverse to a longitudinal axis l1 defined by the handle 1410 of the sheath assembly 1400. The inner sheath 1404 can have an inner lumen 1421 with a distal opening and a proximal opening configured to receive the guidewire 1250 described above. The tapered tip 1420 can have a sharpened tip 1426, with a plurality of cutting teeth 1428.

With reference to FIGS. 28A and 28C, in some embodiments, the sheath assembly 1400 can also include a shorter second dilator or outer sheath 1406 having a distal end 1430 with a beveled tip 1432 and a proximal end 1434 coupled to the handle 1410. The outer sheath 1406 can have a rigid curved profile to provide an access pathway to the vertebrae. With continued reference to FIG. 28A, the outer sheath 1406 can define a longitudinal axis, l2 that is transverse to a longitudinal axis l1 defined by the handle 1410 of the sheath assembly 1400. The proximal end 1434 can also include an elongated opening or slot 1412 on at least a portion of the outer sheath 1406, as described above for receiving various instruments. In some embodiments, the slot 1412 can be an elongate opening that extends from the proximal end 1434 to a distance along the longitudinal length of the outer sheath 1406, as illustrated in FIGS. 28A and 28C. The outer sheath 1406 can also have an inner lumen 1436 with a distal opening and a proximal opening. In some embodiments, the inner diameter of the outer sheath 1406 can be at least approximately 7 millimeters in diameter. In some embodiments, the inner diameter of the outer sheath 1406 can be at least approximately 5 millimeters and/or less than or equal to approximately 20 millimeters.

Various mechanisms can be provided for removably coupling the inner and outer sheaths 1404, 1406 together in a locked configuration in which the distal end 1402 of the inner sheath 1404 extends beyond the distal end 1430 of the outer sheath 1406. In the illustrated embodiments, the inner and outer sheaths 1404, 1406 are coupled together by providing a releasable linking mechanism. The releasable linking mechanism can comprise a spring biased pin that is positioned in the locking member of the inner sheath 1404 and, in a first position, locks the two sheaths 1404, 1406 together. Depressing or sliding a button, moves the pin to release the two sheaths 1404, 1406. With the inner and outer sheaths 1404, 1406 unlocked, the outer sheath 1406 can be advanced over the inner sheath 1404 to expand the access opening. The inner sheath 1404 can then be removed as described above leaving the outer sheath 1406 and its larger inner lumen 1436 in place at the surgery site. In other embodiments, more or fewer dilator tubes can be used. In addition, other access sheaths can be used.

Additional embodiments and/or details of the sheath assembly 1400 can be found in U.S. Patent Publication No. 2006/0030872, filed Aug. 3, 3004 and entitled “Dilation Introducer for Orthopedic Surgery”, which is hereby incorporated by reference herein.

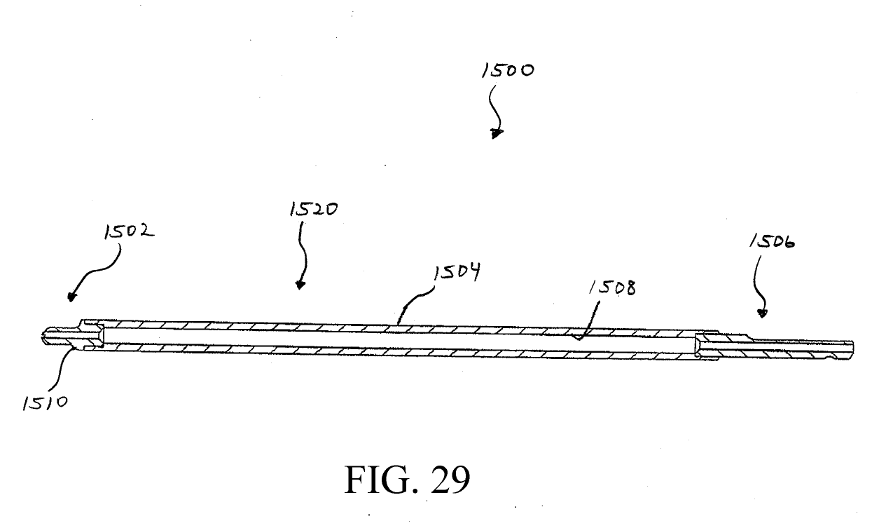

FIG. 29 illustrates an exemplary embodiment of the cortex drill 1500 that was introduced with reference to FIG. 14 above. As mentioned above, the cortex drill 1500 can be used to form a countersink and/or a clearance hole for the fixation device. As shown, the cortex drill 1500 can comprise a body 1504 having a distal end 1502, a proximal end 1506 and a guidewire lumen 1508 extending therethrough. The proximal end 1506 can be configured to engage any of a variety of driving tools. In the illustrated embodiment, the proximal end 1506 has a D-shaped cross-section that can be received within a cavity of a hand held gripping device, which will be described below. In some embodiments, the proximal end can couple with a standard AO quick connect.

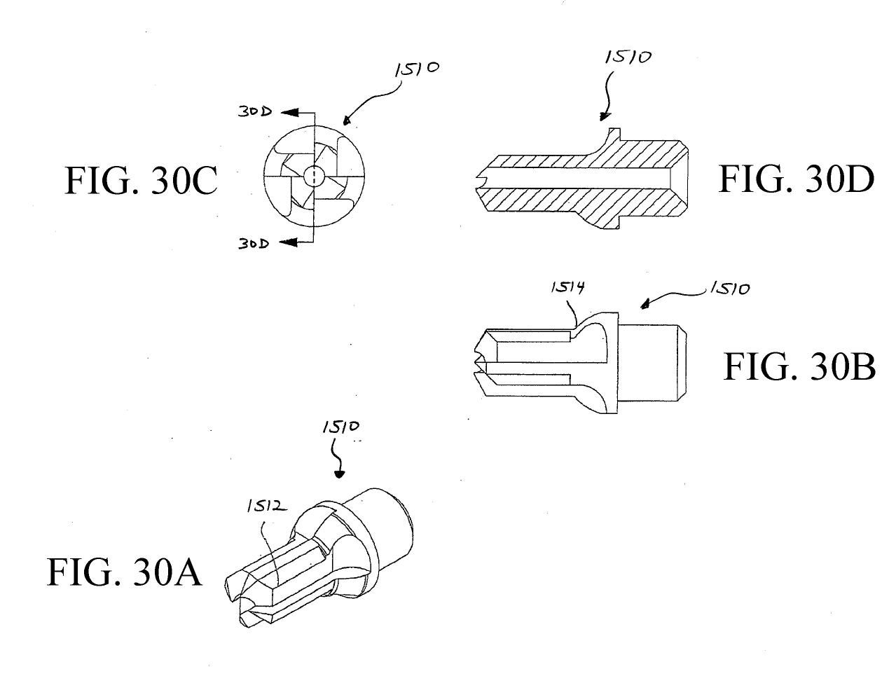

With reference to FIGS. 30A-D, the distal end 1502 of the cortex drill 1500 can be provided with a drilling element 1510 comprising a plurality of cutting elements 1512. In the illustrated embodiment the drilling element 1510 includes four cutting elements 1512. In other embodiments, the drilling element 1510 can include more or fewer than four cutting elements 1512. The cutting elements 1512 can include an outer surface 1514 that preferably generally corresponds to an outer surface profile of the proximal anchor 700 and/or portions of the body 228 of the fixation device 212. The outer surface 1514 can also include one or more removal or cutting features (e.g., flutes, sharp edges, etc.) so as to remove or cut bone as the device cortex drill 1500 is rotated.

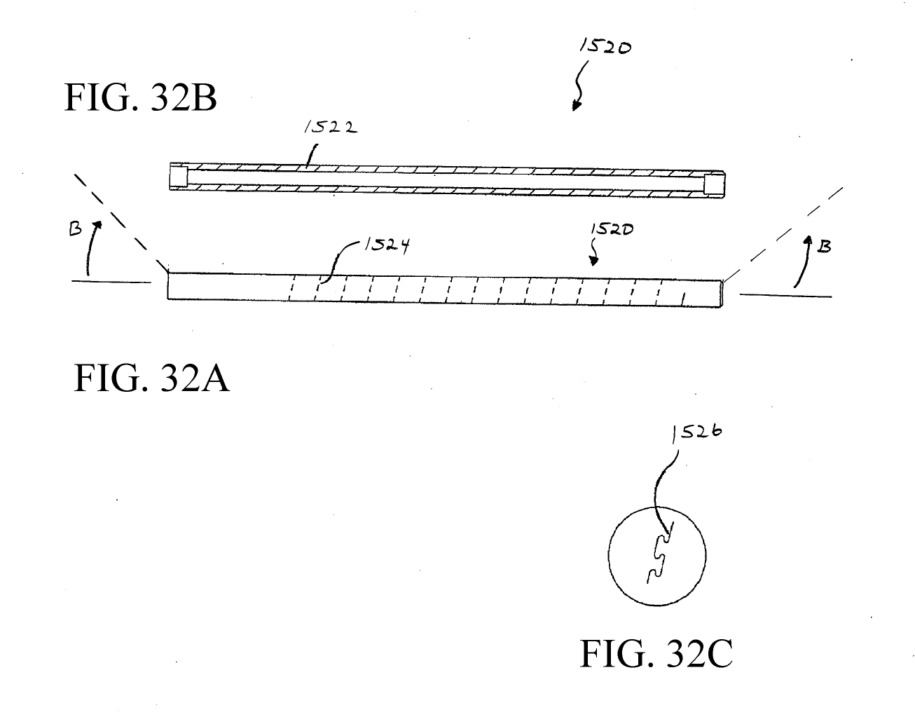

With reference to FIGS. 32A and 32B, in some embodiments, an elongated transmission member 1520 can extend between the proximal end and the distal end of the cortex drill 1500. In the illustrated embodiment, the transmission member 1520 can be bent about its longitudinal axis as indicated by the arrows in FIG. 32A. Thus the transmission member 1520 in some embodiments can be flexible but still capable of transmitting a guiding and/or rotational force to the distal end 1502. In the illustrated embodiment, the transmission member 1520 comprises a tubular wall 1522 in which a generally spiral cut 1524 is formed as is shown in FIGS. 32A and 32C. The spiral cut 1524 can include engaging notches 1526, which facilitate the transmission of rotational force along the tubular wall 1522. In this manner, the transmission member 1520 can be flexible while maintaining sufficient axial force transmission capabilities and can be bent as it is inserted into the sheath assembly 1400 described above. Advantageously, the cortex drill 1500 can be used without or only minimally interfering with the patient's head. As the drill 1500 is bent, it can extend out of the elongated slot 1412 in the sheath assembly 1400. Of course, it is contemplated that other methods can be used to form the flexible transmission member 1520 such as, for example, cuts with different patterns, or transmission members formed of flexible materials such as springs, coils, and/or weaved materials. In some embodiments, the flexible transmission member 1520 can be a cable that is made of wound wires made of durable material, such as metal or plastic.

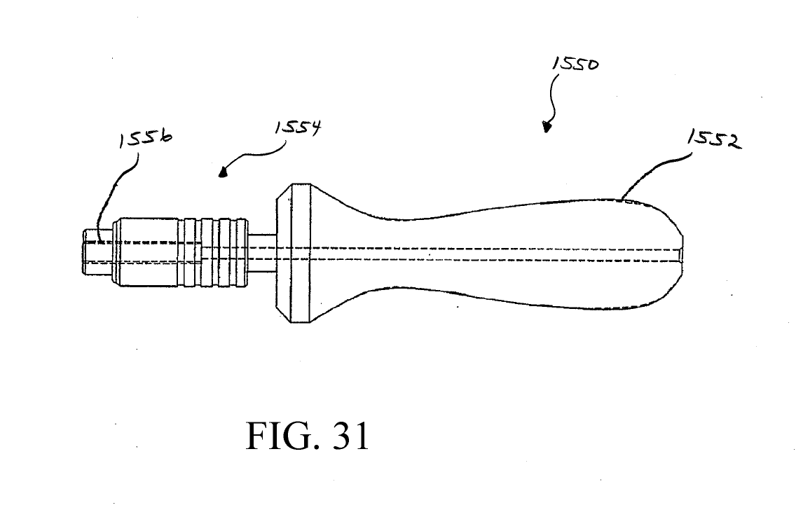

FIG. 31 illustrates a gripping member 1550, which can be coupled to the proximal end 1506 of the cortex drill 1500 described above and to other devices described above. The gripping member 1550 can include a gripping portion 1552 at its proximal end and a distal end 1554. The distal end 1554 includes a cavity 1556 for receiving the proximal end 1506 of the cortex drill 1500. Preferably, the cavity 1556 includes a corresponding shape (e.g., in some embodiments a D-shape to form an AO quick connect with ratcheting features) such that as the gripping member 1550 is rotated, the cortex drill 1500 is also rotated.

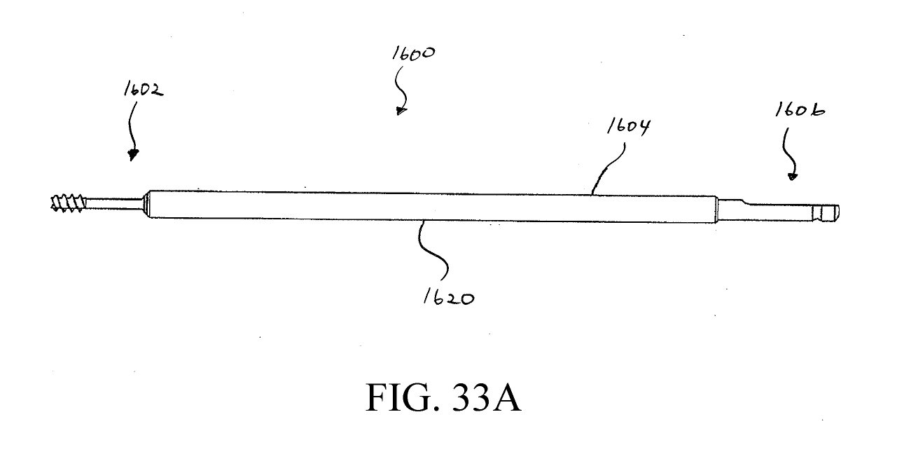

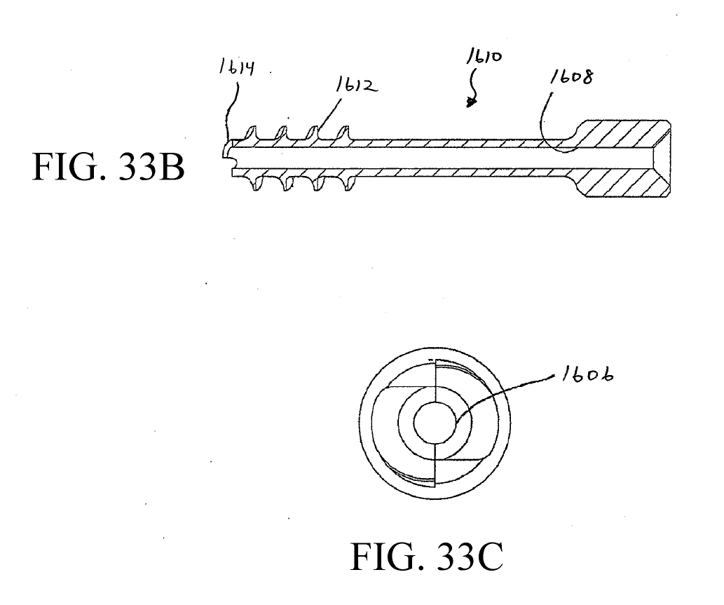

FIGS. 33A-C illustrate an embodiment of the tapping device 1600. As mentioned above, the tapping device 1600 can be cannulated so that it can be inserted over the guidewire 1250 and through the outer sheath 1406 to tap the hole formed in the vertebrae. The tapping device 1600 can comprise a body 1604 having a distal end 1602, a proximal end 1606 and a guidewire lumen 1608 extending therethrough. The proximal end 1606 can be configured to engage any of a variety of driving tools. In the illustrated embodiment, the proximal end 1606 has a D-shaped cross-section that can be received within the cavity 1556 of the hand held gripping member 1550 described above.

With reference to FIGS. 33B-C, the distal end 1602 can be provided with a tapping element 1610 comprising a plurality of threads 1612 and a cutting tip 1614 that correspond to the distal anchor 234 of the fixation device 212. Between the proximal end 1606 and the distal end 1602 of the tapping device 1600 can be an elongated transmission member 1620. In some embodiments, the transmission member 1620 can be flexible about its longitudinal axis as described above with reference to the flexible transmission member 1520 of the cortex drill 1500 illustrated in FIGS. 32A-B. This flexible transmission member 1620 can allow the tapping device 1600 to be advanced through the curvature of the outer sheath 1406. In some embodiments, the transmission member 1620 is configured in a manner similar to the transmission member 1520 described above.

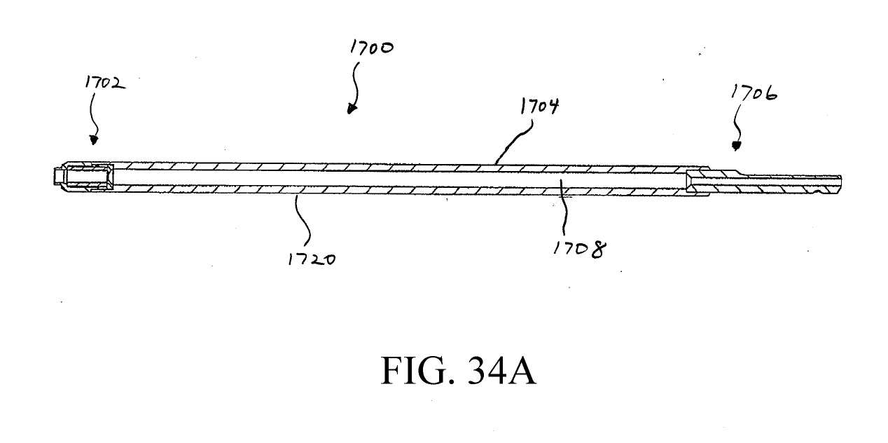

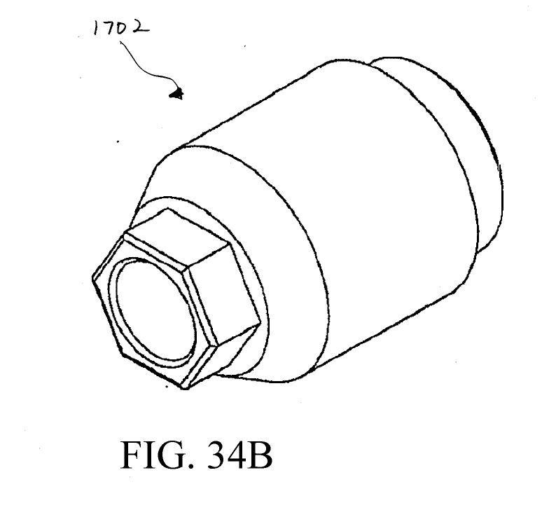

FIGS. 34A-B illustrate a driver 1700 which can be used to drive the fixation device 212 or implant into the vertebrae as described above. In the illustrated embodiment, the driving device 1700 comprises a body 1704 having a distal end 1702, a proximal end 1706 and a guidewire lumen 1708 extending therethrough. The proximal end 1706 can be configured to engage any of a variety of driving tools. In the illustrated embodiment, the proximal end 1706 is has a D-shaped cross-section that can be received within the cavity 1556 of the hand held gripping member 1550 described above and as illustrated in FIG. 31.

With continued reference to FIGS. 34A-B, an outer portion of the distal end 1702 can be configured to engage the gripping structure of the proximal anchor 700. In the illustrated embodiment, the distal end 1702 is hexagonal in shape and configured to be received by a hexagonal recess of the proximal anchor 700. In other embodiments, the distal end 1702 can have any of a variety of different shapes for differently shaped gripping structures on the proximal anchor 700. For example, the distal end 1702 can have a pentagonal shape or any other polygonal shape that is similar to the shape of the gripping structure (e.g., the recess 284) of the proximal anchor 700. In still other embodiments, the distal end 1702 can comprise a recess configured to engage a anti-rotational protrusion formed on the proximal anchor 700.

Between the proximal end and the distal end of the device 1700 can be an elongated transmission member 1720. In the illustrated embodiment, the transmission member 1720 can be bent about its longitudinal axis as described above with reference to the flexible transmission member 1520 of FIGS. 32A-B. The flexible transmission member 1720 can allow the driver 1700 to be advanced through the curvature of the outer sheath 1406.

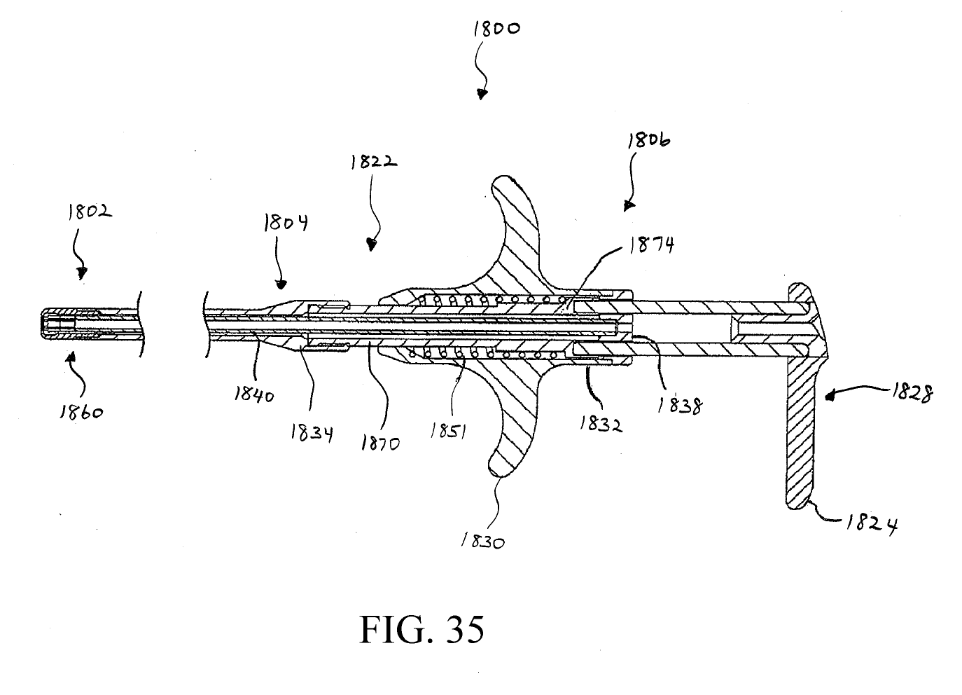

FIG. 35 illustrates the compression device 1800, which can be used to proximally retract the body 228 with respect to the proximal anchor 700 for the fixation device 212 described above. In the illustrated embodiment, the device 1800 generally includes an elongate syringe-shaped body 1822 having a proximal end 1806, and a distal end 1802. The compression device 1800 also generally comprises a plunger 1828 at the proximal end 1806, a finger grip 1830 attached to a proximal housing 1832 located distally from the plunger 1828 and over a connector shaft 1870, and an elongate distal housing 1834 disposed distally of the finger grip 1830. As will be apparent from the description below, the device 1800 preferably defines a lumen that extends through the compression device 1800 such that it may be used over the guidewire 1250.

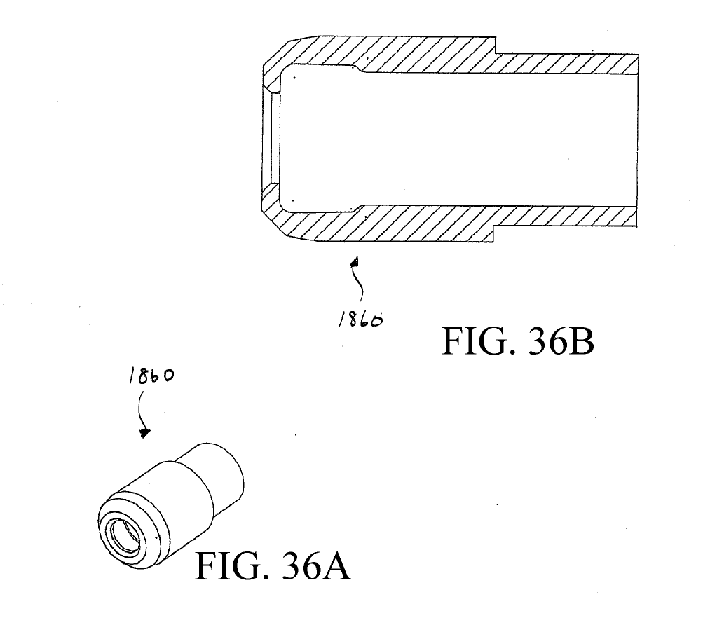

With continued reference to FIG. 35, the illustrated embodiment also includes a tensioner member 1840 that can be disposed within the distal housing 1834 and connector shaft 1870. A distal end of the tensioner member 1840 can be positioned within a distal cap 1860 (see also FIGS. 36A-B). As shown in FIG. 35 and explained below, the distal cap 1860 can be removeably attached to the distal housing 1834 by threads or another removable engagement structure.

As will be explained below, the tensioner member 1840 can be configured to move with the finger grip 1830. The tensioner member 1840 and grip 1830 can move together relative to the plunger 1828, connector shaft 1870 and distal housing 1834. The tensioner member 1840 can desirably be configured to grip a proximal end of the body 228 of the bone fixation device 212. In other embodiments, the connector shaft 1870, distal housing 1834 and the plunger 1828 can be adapted to move together relative to the finger grip 1830 and tensioner member 1840.

With continued reference to FIGS. 35-42B, the plunger 1828, finger grip 1830, distal housing 1834, connector shaft 1870 and tensioner member 1840 can preferably cooperate to cause proximal motion of the tensioner member 1840 relative to the housing 1834 in response to a proximal motion of the finger grip 1830 relative to the plunger 1828. It is contemplated that in other embodiments, many alternative structural arrangements are possible to provide these desired motions, only some of which are described herein.

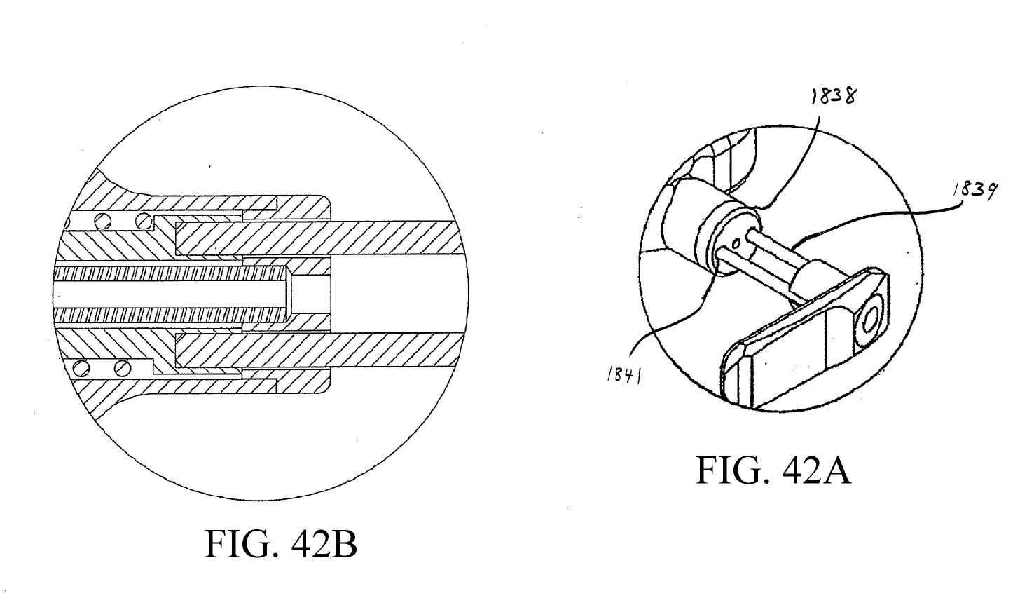

In the illustrated embodiment, the plunger 1824 is attached to the connector shaft 1870 at a proximal end 1874 of the connector shaft 1870. The connector shaft 1870 is connected to the distal housing 1834. As illustrated, the finger grip 1830 is attached to the tensioner member 1840 by coupling the proximal end 1837 of the tensioner member 1840 to the proximal housing plug 1838, which is coupled to the proximal housing 1832 and grip 1830, as illustrated in FIG. 42B. Thus, the finger grip 1830 and tensioner member 1840 can move together and the plunger 1828, connector shaft 1870 and distal housing 1834 can move together. The tensioner member 1840 can slideably engage the distal housing 1834 as the grip 1830 and plunger 1828 are drawn towards each other. As shown in FIG. 42A, the plunger 1828 can be coupled to a proximal end 1874 of the connector shaft 1870 through a pair of prongs 1839, which can extend through openings 1841 formed in the proximal housing plug 1838.

The provision of a tensioner member 1840 on the deployment device 1800 generally allows a clinician to provide proximal retraction to the body 228 of the bone fixation device 212. In the illustrated embodiment, the syringe-shaped body 1822 is generally adapted such that application of a compressive force between the plunger 1828 and the finger grip 1830 results in engagement with a proximal end 230 of the body 228 of the fixation device 212 in order to provide proximal retraction.

As mentioned above, the plunger 1828 is generally adapted to be engaged by the heel of a clinician's hand below the lumen of the device, thus providing a comfortable handle by which the deployment device may be gripped for axial rotation, or a comfortable surface for the compressive force involved in providing retraction to a bone fixation device as described elsewhere herein. It is contemplated that numerous specific arrangements of a plunger (or heel-engagement portion) may be provided according to the particular needs of the clinician. Similarly, the finger grip portion shown and described herein is merely provided by way of example. Other shapes and arrangements are available for providing a finger grip portion.

A biasing member 1851 (e.g., a spring) can be positioned within the proximal housing 1832 to bias the proximal portion 1874 of the connector shaft 1870 in the direction of arrow C in FIG. 35.

In the illustrated embodiment, the plunger 1828 can be held generally stationary and the finger grip 1830 can be pulled towards the plunger 1824. The finger grip 1830 and the tensioner member 1840 can both move proximally relative the plunger 1828 and the distal housing 1834 as the tensioner member 1840 slides along the distal housing 1834. Of course, many other arrangements are possible for providing the desired motion of the tensioner member 1840 relative to the distal housing 1834. For example, a pistol grip can be used. In addition or in combination, the compression device can employ cable and pulley arrangements, levers, or other structures. The various portions may be attached to one another by adhesives, welds, threads, mechanical fasteners, or any other suitable attachment method.

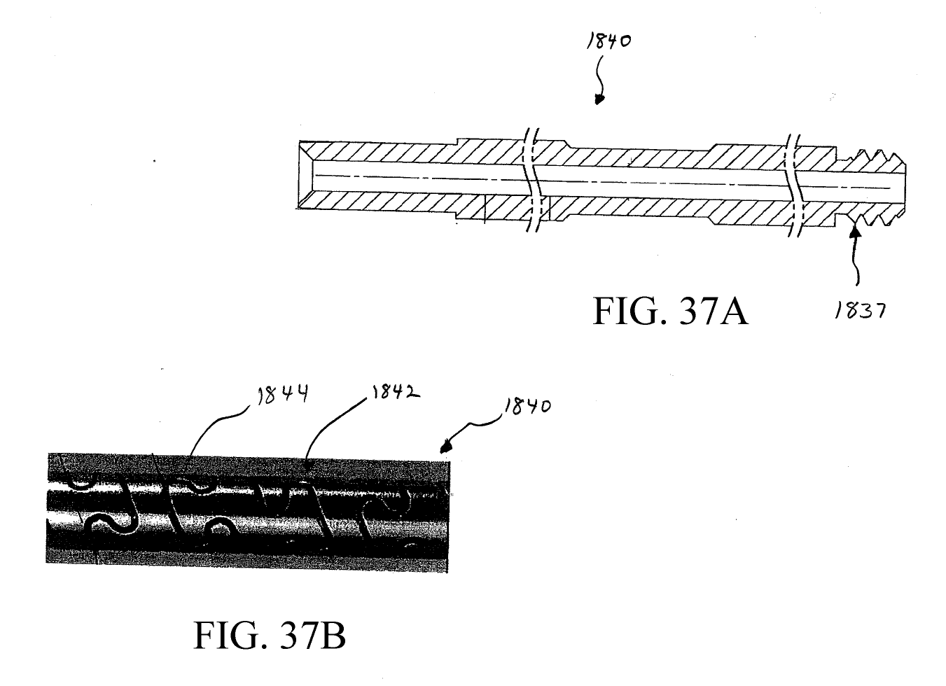

The tensioner member 1840, as illustrated in FIGS. 37A-B, can comprise a solid rod, a hollow tube, one or more cables, or any other appropriate structure such that it functions as described. The tensioner member 1840 can be made of any suitable material such that it has sufficient tensile strength that it will not stretch or otherwise deflect significantly during retraction of the anchor. Suitable materials usable for the construction of a tensioner member 1840 include stainless steel, nylon, etc. and further materials (e.g., metals, plastic and the like). In some embodiments, the tensioner member 1840 can be made of a disposable material, such as plastics. In some embodiments, the tensioner member 1840 can be a flexible elongate member. The flexibility can allow the tensioner member 1840 to be advanced through the curvature of the sheath assembly 1400. Furthermore, the flexible tensioner member 1840 can allow the tensioner member 1840 to be flexed in the direction of arrow A and line 1414 of FIG. 13 to help avoid interference with the patient's head, while a distal end of the tensioner member 1840 maintains a desired position and orientation with respect to the vertebrae. In the illustrated embodiment, the proximal end 1837 of the tensioner member 1840 has threads that are complementary to threads on the proximal housing plug 1838. In other embodiments, the tensioner member 1840 can be attached to the proximal housing plug 1838 through other methods, such as compression fit, adhesives, retaining pins, etc.

In the embodiment illustrated in FIG. 37B, the tensioner member 1840 comprises a tubular wall in which a generally spiral cut pattern 1842 is formed. The spiral cut pattern 1842 can include engaging notches 1844, which facilitate the transmission of axial force along the tubular wall. In this manner, the tensioner member 1840 can be flexible while maintaining sufficient axial force transmission capabilities and can be bent as it is inserted into the sheath assembly 1400 described above. Advantageously, the tensioner member 1840 can be used without or only minimally interfering with the patient's head. As the tensioner member 1840 is bent, it can extend out of the elongated slot 1412 in the sheath assembly 1400. Of course, it is contemplated that other methods can be used to form the flexible tensioner member 1840, such as for example, cuts with different patterns, or transmission members formed of flexible materials such as springs, coils, and/or weaved materials.

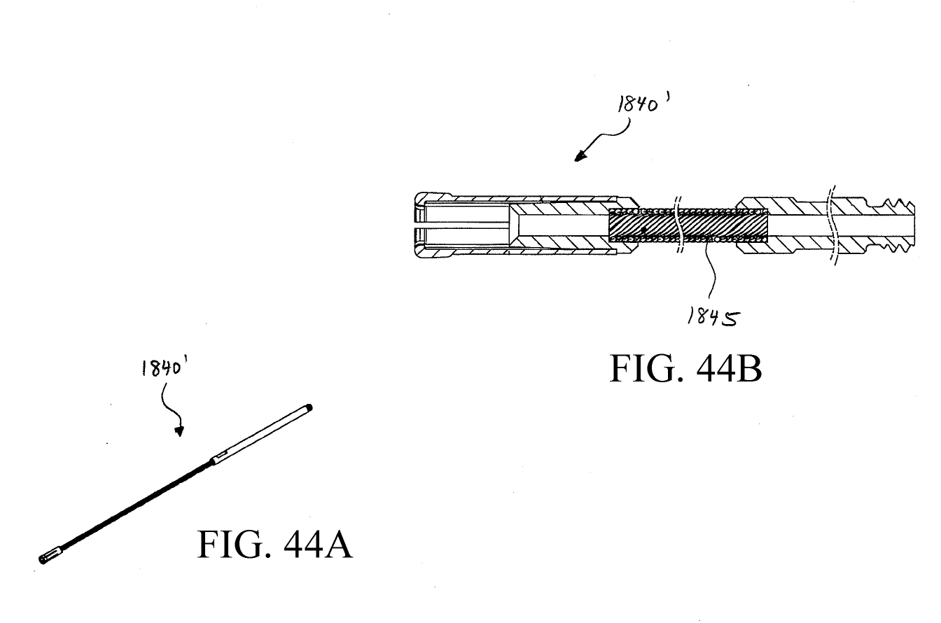

As illustrated in FIG. 44A-B, in alternative embodiments, the tensioner member 1840′ can at least partially include a cable 1845 that is made of wound wires made of durable material, such as metal or plastic. A plastic cable can be used, which can advantageously allow the tensioner member 1840′ to be disposable, or one time use. In the illustrated embodiment, a middle portion of the tensioner member 1840′ is a cable 1845 that is flexible. The cable 1845 can allow the tensioner member 1840′ to be flexible so that it can be bent as it is inserted into the sheath assembly 1400, while maintaining sufficient axial force transmission capabilities. The cable 1845 can be attached to the end components of the tensioner member 1840′ by welds, adhesives, clamps, etc.

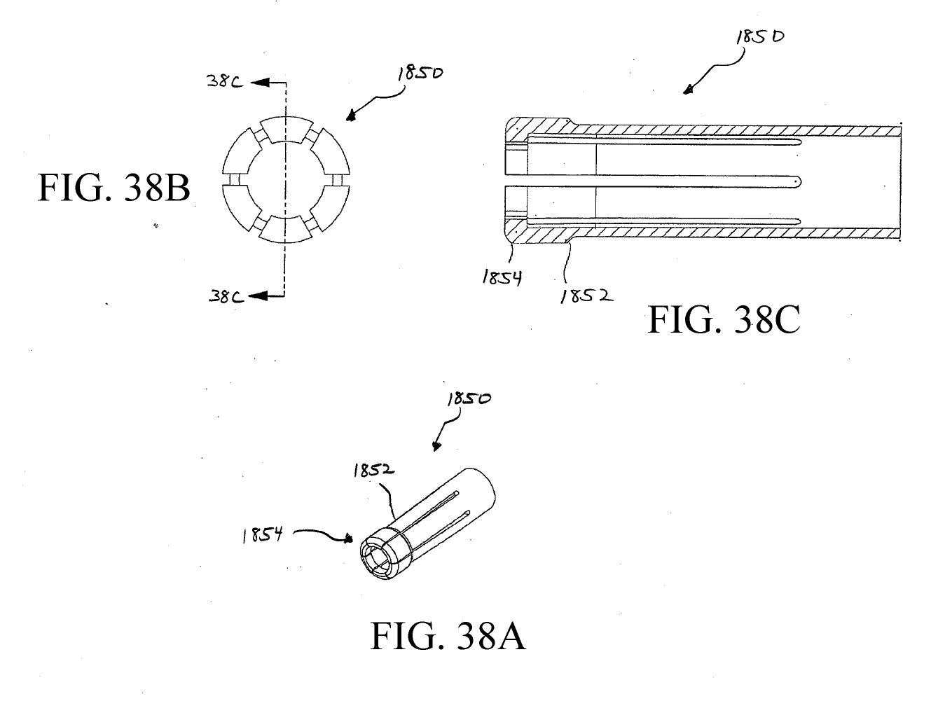

As illustrated in FIGS. 38A-C, the distal end of the tensioner member 1840 can comprises a collet 1850, which can be adapted to be closed around the proximal end 230 of a bone fixation device 212. The collet 1850 can be fixed to the distal end of the tensioner member 1840 by any appropriate methods or devices, or the collet 1850 and tensioner member 1840 can be integrally formed. In some embodiments, the collet 1850 can be threaded onto the distal portion of the tensioner member 1840. Providing a collet with threads advantageously allows collets of varying size to be used interchangeably with a single deployment device 1820 in addition to increasing the ease of cleaning.

In the illustrated embodiment, the collet 1850 comprises a plurality of flexible fingers 1852, each having a gripping head 1854 on its distal end. The flexible fingers 1852 preferably have sufficient tensile strength that the collet 1850 can provide sufficient proximal retraction force to a bone fixation device when the deployment device is operated as described herein.

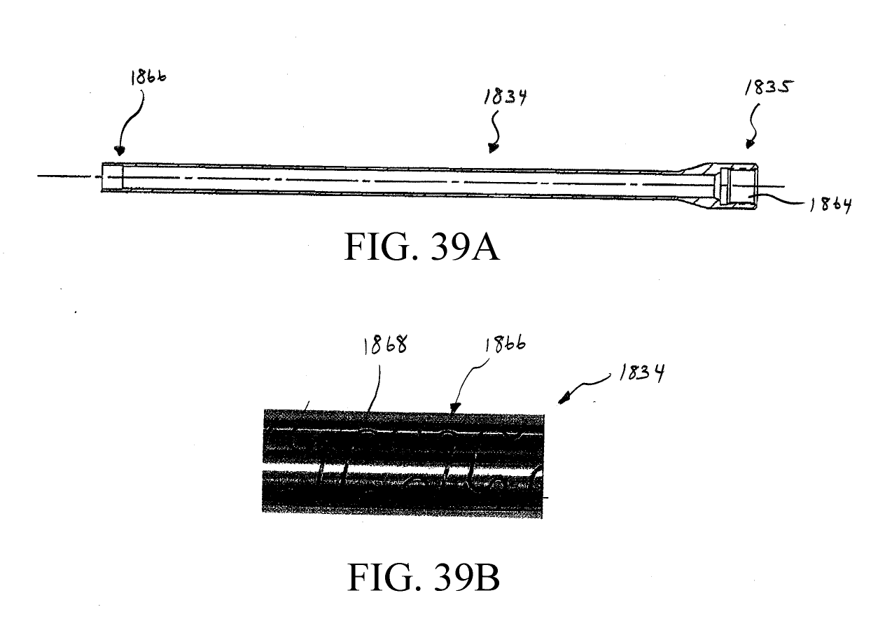

The distal housing 1834, as illustrated in FIG. 39A-B, can comprise a hollow tube, one or more cables, or any other appropriate structure such that it functions as described. The distal housing 1834 can be made of any suitable material such that it has sufficient tensile strength that it will not stretch or otherwise deflect significantly during retraction of the anchor. Suitable materials usable for the construction of a distal housing 1834 include stainless steel, nylon, etc. and further materials (e.g., metals, composites and the like). In some embodiments, the distal housing 1834 can be made of a disposable material, such as plastics. In some embodiments, the distal housing 1834 can be a flexible elongate member. The flexibility can allow the distal housing 1834 to be advanced through the curvature of the sheath assembly 1400. Furthermore, the flexible distal housing 1834 can allow the distal housing 1834 to be flexed in the direction of arrow A and line 1414 of FIG. 13 to help avoid interference with the patient's head, while a distal end of the distal housing 1834 maintains a desired position and orientation with respect to the vertebrae.

The proximal end 1835 of the distal housing 1834 can be configured to couple with a distal end 1872 of the connector shaft 1870. In the illustrated embodiment, the proximal end 1835 has a cavity 1862 for accepting and retaining the distal end 1872 of the connector shaft 1870. In some embodiments, the cavity 1862 can have internal threads for engaging with external threads on the distal end 1872 of the connector shaft 1870. In other embodiments, the distal housing 1834 can be attached to the connector shaft 1870 through other means, such as compression fit, welding, adhesives, retaining pins, etc. Similarly, the distal end 1864 of the distal housing 1834 can be configured to couple with the distal cap 1860. The distal end 1864 can be threaded or otherwise attached, such as by adhesives, welds, etc. to the distal cap 1860.

As illustrated in the embodiment in FIG. 39B, the distal housing 1834 can comprise a tubular wall in which a generally spiral cut pattern 1866 is formed. The spiral cut pattern 1866 can include engaging notches 1868, which facilitate the transmission of axial force along the tubular wall. In this manner, the distal housing 1834 can be flexible while maintaining sufficient axial force transmission capabilities and can be bent as it is inserted into the sheath assembly 1400 described above. Advantageously, the distal housing 1834 can be used without or only minimally interfering with the patient's head. As the distal housing 1834 is bent, it can extend out of the elongated slot 1412 in the sheath assembly 1400. Of course, it is contemplated that other methods can be used to form the flexible distal housing 1834, such as for example, cuts with different patterns, or transmission members formed of flexible materials such as springs, coils, and/or weaved materials. In some embodiments, the distal housing 1834 can be a cable that is made of wound wires made of durable material, such as metal or plastic. A plastic cable can be used, which can advantageously allow the distal housing 1834 to be disposable, or one time use.

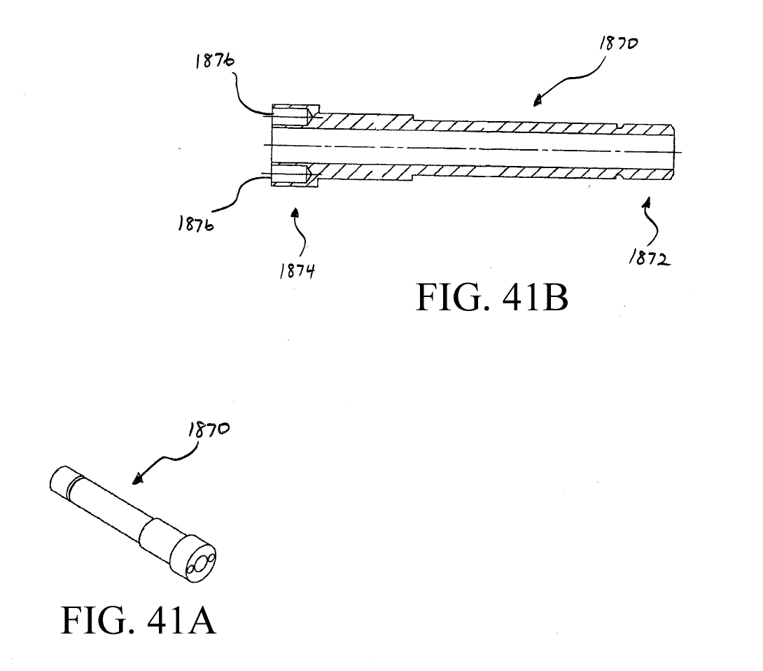

The connector shaft 1870, as illustrated in FIGS. 41A-B, can comprise a hollow tube, one or more cables, or any other appropriate structure such that it functions as described. The connector shaft 1870 can be made of any suitable material such that it has sufficient tensile strength that it will not stretch or otherwise deflect significantly during retraction of the anchor. Suitable materials usable for the construction of a connector shaft 1870 include stainless steel, nylon, etc. and further materials (e.g., metals, plastics, composites and the like). In some embodiments, the connector shaft 1870 can be a rigid member. In other embodiments, the connector shaft 1870 can be a flexible elongate member.

The distal end 1872 of the connector shaft 1870 can be configured to couple with the proximal end 1835 of the distal housing 1834. As described above, the proximal end 1872 can be connected to a cavity 1862 on the distal housing 1834 through threads, press fit, welding, adhesive, etc. The proximal end 1874 of the connector shaft 1870 can be configured to couple with the plunger 1828. In the illustrated embodiment, the proximal end 1874 has prong cavities 1876 for accepting and retaining the prongs 1839 of the plunger 1828. In some embodiments, the prongs 1874 can be attached to the prong cavities 1876 through any means, such as threads, compression fit, welding, adhesives, retaining pins, etc.

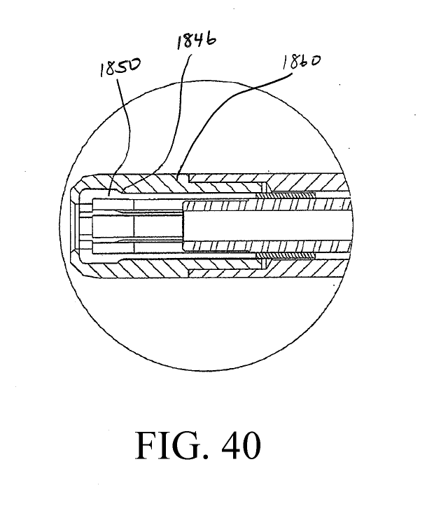

FIG. 40 is a detailed section view of the collet 1850, with a removable distal cap 1860 shown mounted to the distal end of the distal housing 1834. In the illustrated embodiment, a distal portion of the distal cap 1860 has a closing surface 1846 formed by a constriction or reduction in diameter. The closing surface 1846 causes the collet 1850 to close as the distal cap 1860 moves distally relative to the collet 1850. In some embodiments, the closing surfaces 1846 can contact and move the gripping heads 1854 inwardly as the closing surfaces 1846 move distally relative the collet 1850. The closing surface 1846 can alternatively be provided as a constriction in the inner diameter of the distal housing 1834.

As mentioned above, the distal cap 1860 can be threaded or otherwise attached, such as by adhesives, welds, etc. to the distal housing 1834. A removable distal cap, however, can be advantageous in certain embodiments because it allows for greatly simplified cleaning of the deployment device tip. Many embodiments of a distal cap 1860 may be provided depending on the particular application. A distal cap 1860 such as that shown in FIG. 36A, can be provided to abut the flange of the proximal anchor 700 for proximally retracting the anchor as discussed above. Of course in modified embodiments, the distal cap 1860 can include a different shape head or recess as appropriate given the structure of the proximal anchor 700.

In some methods of use, once the distal anchor 234 has been positioned, the finger grip 1830 and plunger 1828 of the compression device 1800 can be compressed, moving the tensioner member 1840 proximally relative to the distal housing 1834 until the gripping heads 1854 engage from the closing surface 1844, thereby causing the gripping heads 1854 to be displaced toward the pin 228. As the tensioner member 1840 continues to be proximally retracted, the gripping heads 1854 eventually engage the proximal flange of the pin 228 thereby allowing the pin 228 and the distal anchor 234 to be pulled proximally relative to the proximal anchor 700. Once the fixation device 212 has been sufficiently retracted, and the superior and inferior vertebrae rigidly coupled together, the second portion of the body 228 can be removed as described below. Modified embodiments, components and/or details of an exemplary embodiment of a compression device can be found in U.S. Pat. No. 7,326,211, issued Feb. 5, 2008, which is hereby incorporated by reference herein in its entirety.

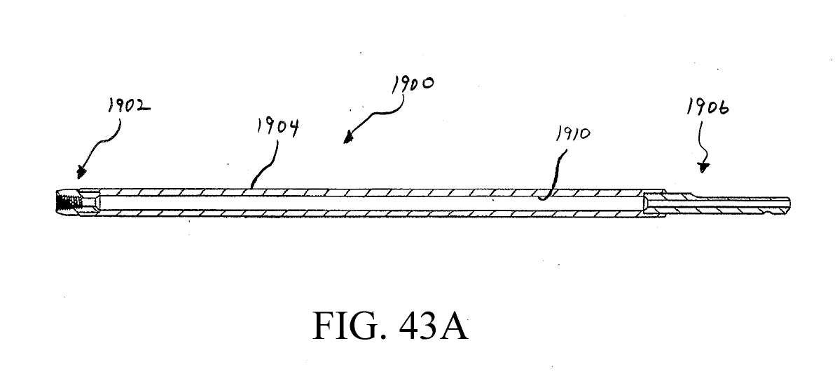

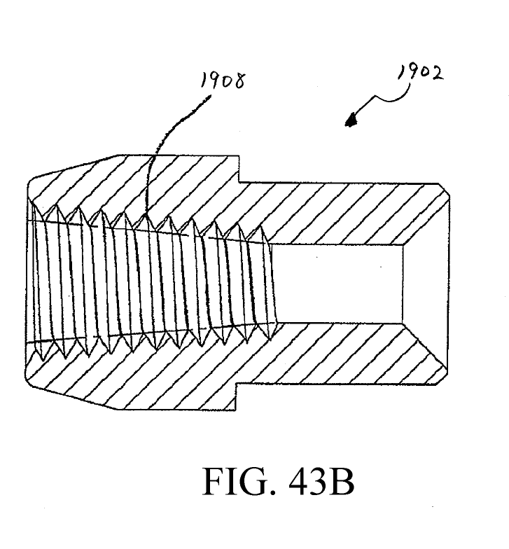

FIGS. 43A-B illustrate an embodiment of the pin remover 1900 that was introduced with reference to FIG. 18 above. As mentioned above, the pin remover 1900 can be inserted over the wire 1250 and through the sheath assembly 1400 to remove a second portion of the body 228 of the fixation device 212. In some embodiments, the pin remover 1900 can be cannulated through its longitudinal length to receive the guidewire 1250. In the illustrated embodiment, the pin remover 1900 comprises a body 1904 having a distal end 1902, a proximal end 1906 and a guidewire lumen 1910 extending therethrough. The proximal end 1906 can be configured to engage any of a variety of driving tools. In the illustrated embodiment, the proximal end 1906 is has a D-shaped cross-section that can be received within the cavity 1556 of the hand held gripping member 1550 described above.

In some embodiments, the body 1904 can bend about its longitudinal axis to advance through the curvature of the sheath assembly 1400, while being able to transmit rotational and axial forces. Furthermore, the flexible body can allow a proximal end 1906 of the pin remover 1900 to be flexed in the direction of arrow A and line 1414 of FIG. 13 while a distal end 1902 of the pin remover 1900 maintains a desired position and orientation with respect to the fixation device 212. In some embodiments, the body 1904 can be configured with spiral cut patterns in a manner similar to the tensioner member 1840 described above.

With reference to FIG. 43B, the distal end 1902 can be provided with a substantially conical threaded cavity 1908. In the illustrated embodiment, the threads of the threaded cavity 1908 are in the opposite direction of the threads that are used to couple the first and second portions of the body 228 of the fixation device. Thus, in use, the distal end 1902 can be advanced through the sheath 1400 until the threaded cavity 1908 engages the coupling 270 on the proximal end 230 of the fixation device 212. Then, by rotating the pin remover 1900 the threads can engage the coupling 270. At a certain point, further rotation between the pin remover 1900 and the coupling 270 are inhibited by the conical nature of the threaded cavity 1908. At this point, further rotations can cause the pull pin 238 of the body 228 to be rotated with respect to the distal anchor 234 causing the distal anchor 234 and pull pin 238 to be decoupled from each other. Once the pull pin 238 is sufficiently decoupled, the pin remover 1900 can be withdrawn to remove the pull pin 238 from the patient.

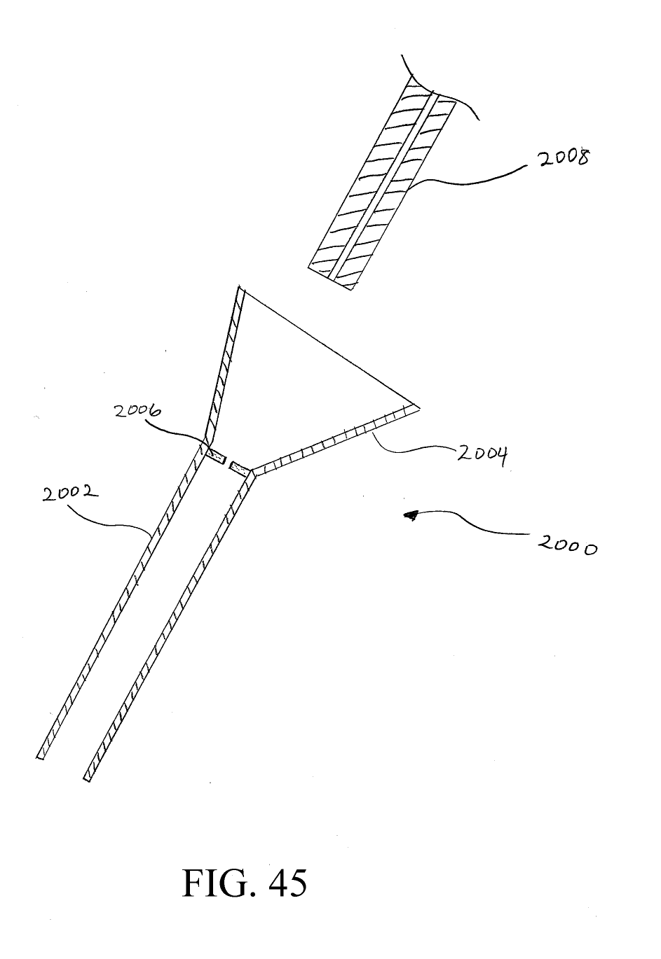

With reference to FIG. 45, the funnel 2000 can comprise a funnel tube 2002 and flared portion 2004. The funnel tube 2002 can have a plug 2006 to help prevent material from escaping proximally out of the funnel tube 2002. The funnel tube 2002 is preferably flexible so that it can be advanced through the curvature of the sheath assembly 1400. Furthermore, the flexible funnel tube 2002 can allow the flared portion 2004 to be flexed in the direction of arrow A and line 1414 of FIG. 13 while the funnel tube 2002 maintains a desired position and orientation inside the sheath assembly 1400. In some embodiments, a push rod 2008 have an outer diameter generally similar to the inner diameter of the funnel tube 2002 can be provided for pushing material through the tube 2002. In some embodiments, the plug 2006 and/or push rod 2008 can be cannulated through its longitudinal length to receive the guidewire 1250.

It should be noted above that the tools above can have dedicated handles instead of interchangeable handles.

The specific dimensions of any of the devices described above can be readily varied depending upon the intended application, as will be apparent to those of skill in the art in view of the disclosure herein. Moreover, although the present invention has been described in terms of certain preferred embodiments, other embodiments of the invention including variations in dimensions, configuration and materials will be apparent to those of skill in the art in view of the disclosure herein. In addition, all features discussed in connection with any embodiment herein can be readily adapted for use in other embodiments herein. The use of different terms or reference numerals for similar features in different embodiments does not imply differences other than those which may be expressly set forth. Accordingly, the present inventions are intended to be described solely by reference to the appended claims, and not limited to the preferred embodiments disclosed herein.

We claim:

1. A device used for deploying a spinal fixation device comprising:
an elongate curved cannulated member having a proximal end, a distal end, a first longitudinal axis extending therebetween, and an outer surface, the cannulated member comprising an elongated opening on the outer surface; and
a handle extending along a second longitudinal axis;
wherein the first and second longitudinal axis form an angle with respect to each other, and
wherein the elongated opening is configured to receive an elongate tubular member having a third longitudinal axis when the third longitudinal axis is oriented transversely to the first longitudinal axis.

2. The device of claim 1, wherein the angle formed by the first and second longitudinal axes is in the range of approximately 30 degrees to approximately 150 degrees.

3. The device of claim 1, wherein the first and second longitudinal axes are substantially perpendicular.

4. The device of claim 1, wherein the elongate tubular member comprises a tool selected from the group of a drill, a tapping member, a driver, a compression device, and a pin removal device.

5. The device of claim 1, wherein the elongate tubular member comprises a flexible member.

6. The device of claim 1, wherein the elongated opening is oriented on the outer surface of the cannulated member facing the housing.

7. A wire introducer for creating a tissue track for a guidewire, the wire introducer comprising:
an elongate curved cannulated member having a first longitudinal axis, a distal end and a proximal end, the distal end including at least one cutting element;
a handle extending along a second longitudinal axis, wherein the first and second longitudinal axes form an angle with respect to each other; and
a flexible trocar having a distal end with a sharpened tip and a proximal end configured to receive a strike pin, the trocar positioned within the cannulated member such that the distal end extends beyond the cannulated member.

8. The wire introducer of claim 7, wherein the trocar and the elongated cannulated member are releasably coupled together by a bayonet connection.

9. The wire introducer of claim 8, wherein the proximal end of the elongated cannulated member comprises a track portion of a bayonet connection.

10. The wire introducer of claim 9, wherein the proximal end of the trocar comprises a pin portion of the bayonet connection.

11. A wire introducer for creating a tissue track for a guidewire, the wire introducer comprising:
an elongate curved cannulated member having a first longitudinal axis, a distal end and a proximal end, the distal end including at least one cutting element and the proximal end configured to receive a strike pin; and
a handle extending along a second longitudinal axis, wherein the first and second longitudinal axes form an angle with respect to each other.

12. The wire introducer as in claim 11, wherein the handle comprises a handle gripping portion that is separated from the elongated curved cannulated member by an elongated extension member.

13. A device used for deploying a spinal fixation device, the device comprising:
an elongate curved flexible transmission member having a distal end and a proximal end;
a tool coupled to the distal end of the transmission member; and
a handle coupled to the proximal end of the transmission member.

14. The device of claim 13, wherein the elongate curved flexible transmission member comprises a flexible cable.

15. A system for coupling a first superior vertebra of a cervical spine to a second inferior vertebra, the system comprising:
a fixation device having a distal end and a proximal end, the distal end configured to extend between the first superior vertebra and the second inferior vertebra; and
an elongate curved tubular device configured to apply the fixation device, the tubular device having a first longitudinal axis and a handle extending along a second longitudinal axis, the first and second longitudinal axes forming an angle with respect to each other such that when the elongated tubular device is applied to the cervical spine from a direction above the cervical spine, the fixation device can be applied without interference from the head of the patient.

16. The system of claim 15, wherein the elongated curved tubular device comprises a curved sheath assembly providing a passage between a first opening above the cervical spine and a second opening positioned adjacent the cervical spine to allow passage of a tool therethrough without interference from the head of the patient.

17. A system for coupling a first superior vertebra of a cervical spine to a second inferior vertebra, the system comprising:
a fixation device having a distal end and a proximal end, the distal end configured to extend between the first superior vertebra and the second inferior vertebra;
at least one curved cannulated device; and
at least one flexible member.

18. The system of claim 17, wherein the at least one curved cannulated device comprises wire introducer.

19. The system of claim 17, wherein the at least one curved cannulated device comprises a dilator sheath.

20. The system of claim 17, wherein the at least one flexible member device comprises a tool configured to drive the fixation device.

21. The system of claim 17, wherein the at least one flexible member comprises a tapping device.

22. The system of claim 17, wherein the at least one flexible member comprises a fascia cutting device.

23. The system of claim 17, wherein the at least one flexible member comprises a drilling device.

24. A method of providing spinal fixation in a cervical spine, the method comprising:
advancing a distal end of an elongate curved cannulated member to a first vertebra in the cervical spine to establish a tissue tract;
advancing a guidewire drill with a generally sharpened distal tip through the first vertebra and into a second vertebra along a first insertion axis;
removing the guidewire drill;
advancing a guidewire with a generally blunt distal tip though the elongate cannulated member and along the first insertion axis into the second vertebra and through a hole created by the guidewire drill;
removing the elongated curved cannulated member;
advancing a curved dilation device over the guidewire; and
inserting a distal end of a fixation device through the dilation device and through the first vertebra and into the second vertebra.

25. The method of claim 24, wherein a trocar is positioned in the elongate curved cannulated member and further comprising the step of removing the trocar from the elongated cannulated member.

26. The method of claim 24, further comprising the step of advancing a deployment device coupled to the fixation device over the second guidewire.

27. The method of claim 24, further comprising the steps of
advancing a fascia cutter with at least one sharp element on a distal end thereof over the guidewire;
cutting a patient's fascia with the fascia cutter;
removing the fascia cutter.

28. A method of inserting a fixation device through a first superior vertebra and into a second inferior vertebra in a cervical portion of the spine, the method comprising the steps of:
advancing a fixation device that comprises a body having a first portion that forms a first bone anchor and a second portion that forms a proximal end through a curved cannulated member and through a portion of the first cervical vertebra;
advancing the bone anchor of the fixation device into the second cervical vertebra;
advancing a proximal anchor distally along the fixation device; and
proximally retracting the body of the fixation device with respect to the proximal anchor to adjust compression across the first and second cervical vertebrae;
with substantially bilateral symmetry, advancing a second fixation device that comprises a body having a first portion that forms a second bone anchor and a second portion that forms a proximal end through a second curved cannulated member and through a portion of the first vertebra;
advancing the bone anchor of the second fixation device into the second vertebra;
advancing a second proximal anchor distally along the second fixation device; and
proximally retracting the body of the second fixation device with respect to the proximal anchor to adjust compression across the first and second vertebrae.

29. A method of providing spinal fixation in a cervical spine, the method comprising:
advancing a distal end of an elongate curved cannulated member to a first, superior vertebrae in the cervical spine;
advancing at least one flexible transmission member through the elongate curved cannulated member; and
inserting a distal end of a fixation device through the elongate curved cannulated member and through the first vertebrae and into the second vertebrae;
wherein the at least one flexible transmission member is used to perform at least one of the following steps:
tap a hole in the cervical spine;
rotate the fixation device; or
apply proximal retraction to a portion of the fixation device.
